(19) United States
(12) Patent Application Publication
(10) Pub. No.: US 2025/0259419 A1
(43) Pub. Date: Aug. 14, 2025

(54) SELECTION OF TARGETS FOR DRUG DEVELOPMENT

(71) Applicant: NUCLEAI LTD., Tel Aviv (IL)

(72) Inventors: Ori Zvi ZELICHOV, Tel Aviv (IL); Orit SHAKED, Ramat Yishai (IL)

(21) Appl. No.: 19/046,960

(22) Filed: Feb. 6, 2025

Related U.S. Application Data
(60) Provisional application No. 63/551,101, filed on Feb. 8, 2024.

(51) Int. Cl. G06V 10/764; G06T 7/00; G06T 7/70; G06V 20/40; G06V 20/69; G16H 30/40

(52) U.S. Cl. CPC G06V 10/764; G06T 7/0012; G06T 7/70; G06V 20/46; G06V 20/69; G16H 30/40

(57) ABSTRACT

Provided herein are methods of drug design comprising using a machine learning model to classify images of tissue samples as being of tissue samples from subject in a first state and tissue samples from subjects in a second state and extracting from the machine learning model groups of biological components comprising at least two biological components whose spatial relationship contributed to the classifying. Methods of drug design, as well as methods of converting a subject from a first state to a second state are also provided.

### CROSS REFERENCE TO RELATED APPLICATIONS

This application claims the benefit of priority of U.S. Provisional Patent Application No. 63/551,101, filed Feb. 8, 2024, the contents of which are incorporated herein by reference in their entirety.

### FIELD OF INVENTION

The present invention is in the field of drug design.

### BACKGROUND OF THE INVENTION

The present invention, in some embodiments thereof, relates to drug development and, more specifically, but not exclusively, to systems and methods for selecting targets for drug development.

Drug development is a time consuming and challenging process. There are a very large number of potential biological targets. Selecting targets which are relevant is challenging. Many drug development processes are based on a trial and error approach, of designing different drugs for different targets, and evaluating the outcomes.

Immunotherapy is an example of a type of drug for treatment of cancer, in which the drug is designed to activate or suppress the immune system. Immune cells may be stimulated to target and destroy abnormal tumor cells in the body of the person.

### SUMMARY OF THE INVENTION

The present invention provides methods of drug design comprising using a machine learning model to classify images of tissue samples as being of tissue samples from subject in a first state and tissue samples from subjects in a second state and extracting from the machine learning model groups of biological components comprising at least two biological components whose spatial relationship contributed to the classifying. Methods of drug design, as well as methods of converting a subject from a first state to a second state are also provided.

Embodiments of the invention may include a method of designing a drug by at least one processor. According to some embodiments, the at least one processor may be configured to obtain at least one image representing protein expression in at least one sample, taken from at least one respective subject; identify, in the at least one image, location of proteins of a first type, within a first type of cells; identify, in the at least one image, location of proteins of a second type, within a second type of cells; and extract, from the at least one image, a spatial feature value representing a spatial relationship between proteins of the first type and proteins of the second type. The at least one processor may subsequently apply a pretrained, Machine Learning (ML) based classification model on data derived from the at least one image, to classify the at least one sample as one of a predetermined set of subject states; and compute a correlation between the spatial feature value and said classification of subject states. Based on the correlation, the at least one processor may select at least one of the first type of proteins and second type of proteins, to design a drug. The drug may be adapted to modulate interaction between proteins of the first type and proteins of the second type.

According to some embodiments, the at least one processor may compute a correlation between the spatial feature value and the state of the subject by applying an interpretability model on the sample classification model, to calculate said correlation as a contribution of the spatial relationship to the classification of the subject's state.

According to some embodiments, the at least one processor may apply an ML based segmentation algorithm on the at least one image, to obtain a plurality of cell segments, each representing a cell in the sample. The at least one processor may subsequently identify one or more cell segments as pertaining to the first cell type or the second cell type, based on indication of protein expression within the one or more cell segments, as depicted in the at least one image.

According to some embodiments, one of the first cell type and second cell type may be a cancer cell, and the other cell type may be an immune cell. Additionally, or alternatively, one of the first cell type and second cell type may be an immune cell and, and the other cell type may be a disease cell, or a healthy cell of the same cell type as said disease cell.

Additionally, or alternatively, the first cell type may be an immune cell type. The at least one processor may be configured to identify one or more cell segments as respective immune cells, belonging to the first cell type; determine a cell activation status value of the immune cells based on indication of protein expression within the respective cell segments; and select at least one of the first type of proteins and second type of proteins further based on the determined cell activation status value.

Additionally, or alternatively, the second cell type may be a cancer cell type. The at least one processor may be configured to identify a plurality of cell segments as respective cancer cells, belonging to the first cell type; define a cluster of cancer cells based on said identification; and select at least one of the first type of proteins and second type of proteins further based on the defined cluster of cancer cells.

According to some embodiments, the spatial feature value may include a measure of at least one of: a distance between a protein of the first type and a protein of the second type, a distribution of distances between proteins of the first type and proteins of the second type, a distance between a cell of the first type and a cell of the second type, a distribution of distances between cells of the first type and cells of the second type, a contact between a protein of the first type and a protein of the second type, a contact between a cell of the first type and a cell of the second type, a structure of a protein of the first type and a protein of the second type, a distribution of proteins of the first type, a distribution of proteins of the second type, an abundance of proteins of the first type, an abundance of proteins of the second type.

Additionally, or alternatively, the spatial relationship comprises a distance metric, indicating a distance between proteins of the first type, and proteins of the second type in the at least one image.

According to some embodiments, the at least one processor may be configured to identify, within the at least one image, a plurality of protein pairs, each comprising a protein of the first type and a protein of the second type, whose locations are within a predetermined distance. The spatial feature value may be defined as an abundance of the protein pairs within the at least one image.

According to some embodiments, the at least one processor may train the ML based classification model by: receiving an annotated training dataset, comprising (i) one or more images of tissue samples taken from subjects, and (ii) corresponding annotations indicating a state of said subjects; and training the sample classification model to determine a state of a subject based on the one or more images, while using the annotations as supervisory information.

According to some embodiments, the at least one image comprises a plurality of protein types, corresponding to a respective plurality of cell types. The ML based classification model may be configured to classify the at least one sample to one of the predetermined set of subject states based on spatial feature values of pairs of protein types, derived from the at least one image.

According to some embodiments, the interpretability model may be configured to identify a pair of protein types, whose spatial feature value statistically significantly contributed to the classification of the state of a subject; and provide one protein type of the pair as the first protein type, and the other protein type of the pair as the second protein type for generating a drug.

Additionally, or alternatively, the interpretability model may be configured to provide a list of pairs of proteins, ordered by the magnitude of the contribution of said spatial feature value to the classification of the state of a subject.

Additionally, or alternatively, the at least one processor may be configured to train the ML based classification model by receiving an annotated training dataset, comprising (i) one or more spatial features, extracted from images of tissue samples taken from subjects, and (ii) corresponding annotations indicating a state of said subjects; and training the classification model to determine a state of a subject based on the one or more spatial features, while using said annotations as supervisory information.

According to some embodiments, the at least one image may be an image of a slide, containing a tissue section, where each protein type may be uniquely stained.

According to some embodiments, the at least one processor may obtain the at least one image by receiving a plurality of slide images representing a tissue section, wherein protein types in each slide may be uniquely stained; and registering the plurality of slide images with each other, to produce a multiplexed image of the tissue section.

Additionally, or alternatively, the at least one image may be a 3-Dimensional (3D) image. The at least one processor may obtain the at least one image by receiving a plurality of slide images representing a respective plurality of tissue sections; and stacking the plurality of slide images to generate the 3D image.

According to some embodiments, the subject states may include, for example a healthy state, a disease state, a state of responding to therapy, a state of non-response to therapy, a state of disease regression, a state of disease stability, a state of disease progression, a state of positive disease prognosis, a state of negative disease prognosis, a state of disease resistance, and a state of disease susceptibility.

According to some embodiments, the distance metric may indicate a distance that surpasses a predetermined threshold. Modulating the interaction between proteins of the first type and proteins of the second type may include, for example, associating between proteins of the first type and proteins of the second type.

Additionally, or alternatively, the distance metric may indicate a distance that is below a predetermined threshold. Modulating the interaction between proteins of the first type and proteins of the second type may include, for example, disrupting an association between proteins of the first type and proteins of the second type.

Embodiments of the invention may include a system for designing a drug. Embodiments of the system may include a non-transitory memory device, wherein modules of instruction code are stored, and at least one processor associated with the memory device, and configured to execute the modules of instruction code.

Upon execution of the modules of instruction code, the at least one processor may be configured to obtain at least one image representing protein expression in at least one sample, taken from at least one respective subject; identify, in the at least one image, location of proteins of a first type, within a first type of cells; identify, in the at least one image, location of proteins of a second type, within a second type of cells; extract, from the at least one image, a spatial feature value representing a spatial relationship between proteins of the first type and proteins of the second type; apply a pretrained, ML-based classification model on data derived from the at least one image, to classify the at least one sample as one of a predetermined set of subject states; compute a correlation between the spatial feature value and said classification of subject states; and based on said correlation, selecting at least one of the first type of proteins and second type of proteins, to design a drug. The drug may be adapted to modulate interaction between proteins of the first type and proteins of the second type.

According to a first aspect, there is provided a method of drug design, the method comprising: receiving, by a trained machine learning (ML) model, one or more images of a tissue sample of a subject, wherein the ML model is trained to distinguish between tissue samples from subjects in a first state and tissue samples from subjects in a second state; classifying the received one or more images as being from a subject in the first state or second state by applying the trained ML model; extracting from the ML model groups of biological components comprising at least two biological components whose spatial relationship contributed to the classifying; thereby identifying at least two biological components for drug design.

According to some embodiments, the trained ML model is produced by a method comprising: receiving one or more extracted spatial features, wherein the spatial features are extracted from a first set of images of tissue samples from subjects in a first state and a second set of images of tissue samples from subjects in a second state, wherein the images contain spatial positions of at least two biological components; generating an annotated training set comprising the extracted spatial relationships and labels corresponding to the extracted spatial relationship, wherein the labels indicate if the spatial relationship was extracted from an image from a subject in the first state or a subject in the second state; training the ML model on the annotated training set to produce a trained ML model capable of distinguishing between images from subjects in the first state and images from subjects in the second state.

According to some embodiments, the method further comprises receiving a first set of images of tissue samples from subjects in a first state and a second set of images of tissue samples from subjects in a second state, wherein the images contain spatial positions of at least two biological components and extracting one of more spatial features from the received one or more images, wherein a spatial feature comprises a spatial relationship between two biological components.

According to some embodiments, the trained ML model is produced by a method comprising: receiving a first set of images of tissue samples from subjects in a first state and a second set of images of tissue samples from subjects in a second state, wherein the images contain spatial positions of at least two biological components; generating an annotated training set comprising the sets of images and labels corresponding to the images, wherein the labels indicate if the images are from a subject in the first state or a subject in the second state; training the ML model on the annotated training set to produce a trained ML model capable of distinguishing between images from subjects in the first state and images from subjects in the second state.

According to some embodiments, the one or more images are images of slides containing a tissue section comprising at least 20 identifiable biological components.

According to some embodiments, the biological components comprise stained biological components, wherein each biological component comprises a unique stain.

According to some embodiments, the biological components comprise at least one nucleic acid molecule comprising a unique barcode, whose spatial position is determined by sequencing or both.

According to some embodiments, the biological components are selected from proteins, nucleic acid molecules, lipids, ions, macromolecule and organelles.

According to some embodiments, the biological components are cell-associated biological components.

According to some embodiments, the images are 3-dimensional images.

According to some embodiments, each of the 3-dimensional images is generated from a plurality of tissue slices, vertically ordered.

According to some embodiments, the received one or more images is from a test subject of unknown state or is from a training set on which the ML model was trained.

According to some embodiments, the first state and second state are selected from: a healthy state and a disease state, a state of responding to therapy and a state of non-response to therapy, a state of disease regression or stability and a state of disease progression, a state of positive disease prognosis and a state of negative disease prognosis and a state of disease resistance and a state of disease susceptibility.

According to some embodiments, the image is an image containing diseased cells or containing healthy cells of the same cell type or tissue type as the diseased cells.

According to some embodiments, the spatial relationship is a measure of at least one of distance between the at least two biological components, contact of the at least two biological components, association of the at least two biological components, structure of the at least two biological components, density of the at least two biological components, abundance of the at least two biological components.

According to some embodiments, the spatial relationship is a measure of distance between the at least two biological components.

According to some embodiments, the spatial relationship is a distribution of distances between the at least two biological components.

According to some embodiments, the group of biological components comprises cellular components.

According to some embodiments, a first component is on a first cell and a second component is on a different second cell and the first component and second component are cell surface components.

According to some embodiments, a first component and a second component are in or on the same cell and the first component and second component are selected from cell surface and intracellular components.

According to some embodiments, the first cell is an immune cell, and the second cell is a disease cell or a healthy cell of the same cell type as the disease cell.

According to some embodiments, at least one of the biological components are cell-specific components that identify a cell type of interest.

According to some embodiments, cell type of interest is a cancer cell and at least one of the biological components is a tumor-specific antigen.

According to some embodiments, the extracting comprises employing an interpretability model.

According to some embodiments, the interpretability model outputs pairs of biological components whose spatial relationship statistically significantly contributed to the classifying.

According to some embodiments, the interpretability model outputs a list of pairs ordered by the ability of the magnitude of the contribution of the spatial relationship, the magnitude of the significance of the spatial relationship or both.

According to some embodiments, extracting one or more spatial features from the one or more received images comprises assembling a table of spatial features and measures for each spatial for each image.

According to some embodiments, the extracting is via an ML algorithm.

According to some embodiments, the method further comprises designing a drug that alters a spatial relationship of the pair of biological components.

According to some embodiments, the second state is an undesired state, and the first state is a desired state, and (a) the pair of biological components are closer to each other in the second state than the first state and the drug disrupts the association of the pair of biological components; or (b) the pair of biological components are closer to each other in the first state than the second state and the drug causes association of the pair of biological components to each other.

According to some embodiments, causing association comprises binding at least one of the biological components and bringing it to a disease site thereby increasing its abundance in the disease site.

According to some embodiments, the method further comprises administering the designed drug to a subject in the second state.

According to another aspect, there is provided a method of generating an agent, the method comprising identifying at least two biological components by a method of the invention, and (a) if the at least two biological components are closer to each other in the second state than the first state designing a drug that disrupts the association of the pair of biological components; or (b) if the at least two biological components are closer to each other in the first state than the second state designing a drug that causes association of the pair of biological components.

According to some embodiments, the generated agent is for use in converting a subject is a second state into a subject in a first state,

According to another aspect, there is provided a method of converting a subject in a second state into a subject in a first state, the method comprising administering to the subject a drug that disrupts association of a pair of biological components or causes association of a pair of biological components, wherein the pair of biological components is selected by a method of the invention.

According to some embodiments, a drug that causes association is a multi-specific molecule that binds each of the at least two biological components.

According to some embodiments, the multi-specific molecule is a bispecific antibody.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1 is a block diagram of a system for identifying two or more expressed structures for developing a drug for treating subjects suffering from a medical condition, in accordance with some embodiments of the present invention;

FIG. 2 is a flowchart of a method of identifying two or more expressed structures for developing a drug for treating subjects suffering from a medical condition, in accordance with some embodiments of the present invention;

FIG. 3 is a flowchart of a method for training a machine learning model that generates an outcome of a target medical state of a person in response to an input of spatial features, in accordance with some embodiments of the present invention;

FIG. 4 is a flowchart of another exemplary approach for identifying two or more expressed structures for developing a drug for treating subjects suffering from a medical condition, in accordance with some embodiments of the present invention;

FIG. 5 is a flowchart of an exemplary high level process for identifying two or more expressed structures for developing a drug for treating subjects suffering from a medical condition, in accordance with some embodiments of the present invention;

FIG. 6 is a flowchart of an exemplary process for developing drugs for treating subjects suffering from a medical condition, in accordance with some embodiments of the present invention;

FIG. 7 is a block diagram depicting a computing device, which may be included within an embodiment of a system for designing a drug, according to some embodiments;

FIG. 8 is a block diagram depicting a system for designing a drug, according to some embodiments of the invention; and

FIG. 9 is a flow diagram depicting a method of designing a drug by at least one processor, according to some embodiments of the invention.

### DETAILED DESCRIPTION OF THE INVENTION

The present invention, in some embodiments thereof, relates to drug development and, more specifically, but not exclusively, to systems and methods for selecting targets for drug development.

An aspect of some embodiments of the present invention relates to systems, methods, an apparatus, and/or code instructions (e.g., stored on a memory and executable by one or more hardware processors) for identifying one or more expressed structures for developing a drug for treating subjects. Spatial features are extracted from image(s) of slide(s) depicting at least a portion of a target tissue of an individual. The spatial features are based on physical distances between different expressed structures in the tissue, for example, average distance between a specific cell and a specific protein, or a distance between a tumor cell and an immune cell. The spatial features are fed into a machine learning (ML) model. The ML model is trained on a training dataset of records, where each record includes sample image(s) of sample slide(s) depicting at least a portion of a sample tissue of a sample individual, and a ground truth label indicating a medical state of the sample individual. An outcome of a target medical state may be obtaining from the ML model. Target spatial feature(s) of the spatial features fed into the ML model, that statistically significantly contributed to the outcome, are obtained by applying an interpretability model to the ML model. One or more expressed structure(s) corresponding to the target spatial feature are identified. Examples of the expressed structure(s) include, but are not limited to: expressed protein, cell type, genomic structures, RNA, DNA, methylation, transcriptomic structure, visually distinguishable structure identified based on a staining of the slide based on immunohistochemistry and/or multiplexed immunohistochemistry, and visually distinguishable structure identified based on immunofluorescence. The identified target spatial feature(s) are used to design a drug to target the expressed structure(s) corresponding to the target spatial feature. For example, the drug may be designed to engage the two expresses structures by bringing the two expressed structures close to one another, the drug may be designed to disengage the two expressed structures by increasing a distance between the two expressed structures, and/or the drug may be designed to increase a first expressed structure within a second expressed structure by bringing the first expressed structure to within and/or in proximity to the second expressed structure (e.g., increase a specific protein in tumor cells).

By another aspect, there is provided a method of drug design, the method comprising: receiving, by a trained machine learning (ML) model, one or more images of a tissue sample of a subject; classifying the received one or more images as being from a subject in a first state or a second state by applying the trained ML model; and extracting from the ML model groups of components comprising at least two components whose spatial relationship contributed to the classifying; thereby identifying at least two components for drug design.

In some embodiments, the method is an in vitro method. In some embodiments, the method is an ex vivo method. In some embodiments, the method is a method of selecting components for drug design. In some embodiments, the method is a method of selecting targets for drug design. In some embodiments, the method is a method of selecting targets for drug design. In some embodiments, a target is a pair of targets. In some embodiments, a target is a component pair. In some embodiments, the components are biological components. In some embodiments, the components are spatial features. In some embodiments, a spatial feature comprises the spatial relationship between two components. In some embodiments, a spatial feature is a spatial relationship. In some embodiments, the spatial feature contributed to the classifying.

In some embodiments, the components are biological components. In some embodiments, the components are organic components. In some embodiments, the components are cell-associated components. In some embodiments, the components are cellular components. In some embodiments, the components are not soluble components. In some embodiments, the components are attached to or within a cell. In some embodiments, the components are in an intercellular region. In some embodiments, the components are not in an intercellular region. In some embodiments, the components are in intercellular vesicles. In some embodiments, the components are not in intercellular vesicles. In some embodiments, the components are in secreted vesicles. In some embodiments, the components are not in secreted vesicles. In some embodiments, in is encapsulated in. In some embodiments, in is embedded in a membrane of.

In some embodiments, the components are disease components. In some embodiments, the components are components of a diseased cell, tissue or cell type. In some embodiments, the components are unique or upregulated in a disease cell, tissue or cell type. In some embodiments, the components are immune components. In some embodiments, the components are components of an immune cell. In some embodiments, the components are involved in immune signaling. In some embodiments, the components are not disease components. In some embodiments, the components are found on healthy cells. In some embodiments, the components are found in healthy tissue. In some embodiments, the components are found on healthy and nonhealthy cells. In some embodiments, the components are found in healthy and nonhealthy tissue. In some embodiments, the components are found on healthy and diseased cells. In some embodiments, the components are found in healthy and diseased tissue. In some embodiments, the components are not disease specific. In some embodiments, the components are found in the first state and the second state. In some embodiments, the components are found on cells of the first state. In some embodiments, the components are found in tissue of the first state. In some embodiments, the components are found on cells of the second state. In some embodiments, the components are found in tissue of the second state. In some embodiments, the components are found on cells of the first state and cells of the second state. In some embodiments, the components are found in tissue of the first state and tissue of the second state.

In some embodiments, a group of components is at least 2 components. In some embodiments, a group of components is at least a pair of components. In some embodiments, a group of components is a pair of components. In some embodiments, components are at least 2, 3, 4, 5, 6, 7, 8, 9, 10, 15, 20, 25, 30, 35, 40, 45, 50, 60, 70, 75, 80, 90 or 100 components. Each possibility represents a separate embodiment of the invention. In some embodiments, the components are at least 20 components. In some embodiments, the image comprises at least 2, 3, 4, 5, 6, 7, 8, 9, 10, 15, 20, 25, 30, 35, 40, 45, 50, 60, 70, 75, 80, 90 or 100 components. Each possibility represents a separate embodiment of the invention. In some embodiments, the image comprises at least 20 components. In some embodiments, the tissue comprises at least 2, 3, 4, 5, 6, 7, 8, 9, 10, 15, 20, 25, 30, 35, 40, 45, 50, 60, 70, 75, 80, 90 or 100 components. Each possibility represents a separate embodiment of the invention. In some embodiments, the tissue comprises at least 20 components. In some embodiments, the tissue comprises at least 2 components.

In some embodiments, components are at most 50, 60, 70, 75, 80, 90, 100, 150, 200, 250, 300, 350, 400, 450, 500, 600, 700, 800, 900, 1000, 5000, 10000, 15000, 20000, 25000, 30000, 40000, 50000, 60000, 70000, 80000, 90000 or 100000 components. Each possibility represents a separate embodiment of the invention. In some embodiments, the image comprises at most 50, 60, 70, 75, 80, 90, 100, 150, 200, 250, 300, 350, 400, 450, 500, 600, 700, 800, 900, 1000, 5000, 10000, 15000, 20000, 25000, 30000, 40000, 50000, 60000, 70000, 80000, 90000 or 100000 components. Each possibility represents a separate embodiment of the invention. In some embodiments, the tissue comprises at most 50, 60, 70, 75, 80, 90, 100, 150, 200, 250, 300, 350, 400, 450, 500, 600, 700, 800, 900, 1000, 5000, 10000, 15000, 20000, 25000, 30000, 40000, 50000, 60000, 70000, 80000, 90000 or 100000 components. Each possibility represents a separate embodiment of the invention. It will be understood by a skilled artisan that the number of components to be examined is limited only by the detection technology and that any number of markers could be theoretically examined in a single image. Simultaneous identification/analysis of components in a given image is thus limited only by the technology available. In some embodiments, the component is protein and there are at most 100 components. In some embodiments, the component is RNA and there are at most 20000 components. It will be understood that current technology allows for multiplexing of more RNAs than proteins, but regardless any number of components can be multiplexed if it is within the capability of the detection technology.

In some embodiments, components are identifiable components. In some embodiments, components are tagged components. In some embodiments, components are labeled components. In some embodiments, components are uniquely identifiable components in the at least one images. In some embodiments, identifiable is visually identifiable. In some embodiments, identifiable is identifiable by microscopy. Methods of microscopic analysis of images including determining the location of specific components are well known in the art and any such method may be employed. In some embodiments, identifiable is identifiable my sequencing. In situ sequencing that retains spatial positioning is known in the art and methods for performing such spatial sequencing are also known. Any such method may be employed.

In some embodiments, the components are selected from proteins, nucleic acid molecules, lipids, ions, macromolecules and organelles. In some embodiments, the components are proteins. In some embodiments, the components are nucleic acid molecules. In some embodiments, the components are lipids. In some embodiments, the components are ions. In some embodiments, the components are macromolecules. In some embodiments, the components are organelles.

In some embodiments, the components are proteins. In some embodiments, the proteins are cell associated proteins. In some embodiments, the proteins are secreted proteins. In some embodiments, the proteins are not secreted proteins. In some embodiments, the proteins are surface proteins. In some embodiments, the proteins are surface markers. In some embodiments, the proteins are intracellular proteins. In some embodiments, the proteins are receptors. In some embodiments, the proteins are involved in immune response. In some embodiments, the proteins are immune proteins. In some embodiments, the proteins are immune response proteins. In some embodiments, the proteins are components of an immune synapse. In some embodiments, the protein is an immune checkpoint protein. In some embodiments, the protein is involved in immune checkpoint signaling.

In some embodiments, the proteins are disease proteins. In some embodiments, the proteins are expressed by diseased cells. In some embodiments, the proteins are overexpressed by diseased cells. In some embodiments, the proteins are unique to diseased cells. In some embodiments, the proteins are peptides. In some embodiments, the proteins are protein fragments. In some embodiments, the proteins are cancer cell antigens. In some embodiments, the proteins are cancer specific antigens.

In some embodiments, the disease is a proliferative disease. In some embodiments, the disease is cancer. In some embodiments, the cancer is a solid cancer. In some embodiments, the cancer is a tumor. In some embodiments, the cancer is within a tissue. In some embodiments, the cancer is colorectal cancer. In some embodiments, the cancer is lung cancer. In some embodiments, the lung cancer is non-small cell lung cancer (NSCLC). In some embodiments, the cancer is skin cancer. In some embodiments, the skin cancer is melanoma. In some embodiments, the disease is a disease treatable by immunotherapy. In some embodiments, the disease is a disease treatable by an enhanced immune response.

As used herein, the terms “peptide”, “polypeptide” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide” and “protein” apply to naturally occurring amino acid polymers. In another embodiment, the terms “peptide”, “polypeptide” and “protein” apply to amino acid polymers in which one or more amino acid residue is an artificial chemical analogue of a corresponding naturally occurring amino acid. Methods of identifying protein location are well known in the art, the include immunohistochemistry, immunostaining, fluorescent staining, and many others. Any method of protein identification, localization, and/or visualization may be used.

In some embodiments, the components are nucleic acid molecules. In some embodiments, the nucleic acid is DNA. In some embodiments, the DNA is genomic DNA. In some embodiments, the nucleic acid is RNA. In some embodiments, the RNA is messenger RNA. In some embodiments, the RNA is a non-coding RNA. In some embodiments, the nucleic acid molecule is cytoplasmic. In some embodiments, the nucleic acid molecule is nuclear. In some embodiments, the nucleic acid molecule is extracellular. In some embodiments, the nucleic acid molecule is vesicle bound. mRNA can be used in a similar manner to protein as it indicates the expression of a target gene. Methods of measuring nucleic acid molecules are well known in the art and any such method may be used. Examples of such methods include, but are not limited to in situ hybridization, spatial nucleotide capture, and in situ spatially resolved sequencing.

In some embodiments, the nucleic acid molecule comprises a barcode. In some embodiments, the barcode is a nucleotide barcode. Barcodes are well known in the art and any appropriate sequence or moiety may be used. In some embodiments, the barcode is a sequence not found in nature. In some embodiments, the barcode is a sequence not found in the tissue. In some embodiments, the barcode is a sequence not found in cells in the image. In some embodiments, the barcode is a unique barcode. In some embodiments, each component comprises a unique barcode. In some embodiments, each component is labeled by a unique barcode. In some embodiments, the spatial position is determined by sequencing. In some embodiments, the sequencing is next generation sequencing. In some embodiments, the sequencing is massively parallel sequencing. In some embodiments, the sequencing is in situ sequencing. In some embodiments, the sequencing is spatially conserved sequencing. In some embodiments, the sequencing is sequencing of the barcode.

The terms “nucleic acid molecule” include but not limited to single-stranded RNA (ssRNA), double-stranded RNA (dsRNA), single-stranded DNA (ssDNA), double-stranded DNA (dsDNA), small RNA such as miRNA, siRNA and other short interfering nucleic acids, snoRNAs, snRNAs, tRNA, piRNA, tnRNA, small rRNA, hnRNA, lncRNA, circulating nucleic acids, fragments of genomic DNA or RNA, degraded nucleic acids, ribozymes, viral RNA or DNA, nucleic acids of infectious origin, amplification products, modified nucleic acids, plasmidical or organellar nucleic acids and artificial nucleic acids such as oligonucleotides.

In some embodiments, the first component is on a first cell and the second component is on a second cell. In some embodiments, the first and second cell are different cells. In some embodiments, the first and second cells are the same cell. In some embodiments, the same cell is the same cell type. In some embodiments, different cells are different cell types. In some embodiments, the first and second components are on different cells and the components are cell surface components. In some embodiments, the first and second components are the same cell or cell type, and components are cell surface or intracellular components. In some embodiments, the first cell is of one cell type and the second cell is of a different cell type. In some embodiments, the first cell is an immune cell, and the second cell is a disease cell. In some embodiments, the first cell is an immune cell, and the second cell is a healthy cell of the same cell type as the disease cell. In some embodiments, the first cell is an immune cell, and the second cell is a healthy cell of the same tissue as the disease cell.

At least some implementations of the systems, methods, apparatus, and/or code instructions described herein relate to the technical and/or medical problem of selecting targets for development of drug(s) for treating subjects suffering from a medical condition, for example, cancer, auto-immune, neurology, ophthalmology, infectious, metabolic, hematologic, and tissue repair. At least some implementations of the systems, methods, apparatus, and/or code instructions described herein improve the technical field of design of new drugs, by improving the process of selecting targets for the drugs. Standard molecular approaches for discovery of targets for design of drugs include, for example, DNA sequences, RNA sequences, or proteomics, are mostly genetic, based on clinical knowledge and/or common knowledge regarding immune response. Such standard approaches do not consider spatial context. In contrast, at least some implementations of the systems, methods, apparatus, and/or code instructions described herein consider spatial locations of targets in tissue samples and/or specific protein interactions for immune response. Specific interaction(s) between different immune and/or tumor proteins and/or specific location(s) of cells in the tumor may be considered in selecting the targets for design of drugs.

In at least some implementations, the solution to the technical problem, and/or the technical improvement, is obtained by identifying the spatial cell and/or protein interactions for immune response, by analyzing, for example, multiplex-stained histopathology slides of subjects treated with drugs.

It should be noted that selection of spatial features extracted from images of slides of pathological tissue used for selection of targets for design of drugs are different than selection of other features extracted from the images of slides of pathological tissue for training a machine learning model that generates automated diagnoses and/or selects treatments. The spatial features that are useful for selection of targets for design of drugs are not necessarily useful for training the machine learning model that generates automated diagnoses and/or selects treatments, since, for example, the spatial features are not necessarily significantly correlated with diagnosis and/or clinical outcomes.

The approaches described herein for automated selection of spatial features extracted from images of slides of pathological tissue used for selection of targets for design of drugs, cannot be performed using manual approaches. A user (e.g., pathologist), viewing slides under a microscope, cannot visually and/or manually determine which spatial features are relevant for selection of targets for design of drugs. Moreover, the user may not be able to manually extract spatial features, for example, many spatial features require complex computations to determine their value, such as graph based and/or cluster-based computations, which cannot be manually performed by a user.

For example, in a population of patients that are sensitive to immunotherapy, there may be two known proteins from two different cell types (e.g., Tumor cell and T-helper cell) that are commonly present near each other. This immune “synapse” might be responsible for the immune response and may be identified as a spatial feature used to design an immune-oncology drug that targets the proteins of the Tumor cell and T-helper cell. The spatial feature cannot be identified as being an important target for development of immune-oncology drugs using other non-spatial omics. Moreover, the spatial feature would not necessarily be identified as an important target for development of immune-oncology drugs in other approaches that use extracted feature to train ML models to generate outcomes indicating likely diagnosis and/or suggest use of existing drugs for treatment and/or predict clinical outcomes. The discovery of the spatial feature may be used to design drugs that engage the two target proteins, to create the immune synapse and destroy the tumor with or without the addition of other, approved immune checkpoint inhibitor.

In another example, in a population of patients that are resistant to immunotherapy there may be two known proteins from two different cell types (e.g., macrophages and Treg cells) that are commonly present near each other. This immune “synapse” might be responsible for the immune resistance and may not be identified as a spatial feature used to design an immune-oncology drug that targets the proteins of the macrophages and Treg cells. The spatial feature cannot be identified as being an important target for development of immune-oncology drugs using other non-spatial omics. Moreover, the spatial feature would not necessarily be identified as an important target for development of immune-oncology drugs in other approaches that use extracted feature to train ML models to generate outcomes indicating likely diagnosis and/or suggest use of existing drugs for treatment and/or predict clinical outcomes. The discovery of the spatial feature may be used to design drugs that disengage the two target proteins, block the communication between the cells and reprogram the tumor from being resistant to being sensitive.

In some embodiments, the one or more images are images of slides. In some embodiments, the one or more images are images of tissue. In some embodiments, the tissue is a tissue section. In some embodiments, the tissue is a tissue slice. In some embodiments, a slice is a single layer of cells. In some embodiments, the tissue is a 3-D tissue. In some embodiments, the images are 3-dimensional images. In some embodiments, the tissue comprises a plurality of layers of cells. In some embodiments, the images are generated from a plurality of slices of tissue virtually ordered. In some embodiments, the images are a Z-stack of slices of tissue. In some embodiments, the images are generated from a Z-stack of images of slices of tissue. In some embodiments, each cell in the image can be distinctly resolved. In some embodiments, each cell in the image can be distinctly identified. In some embodiments, the one or more images are computer files. In some embodiments, the one or more images are image files. In some embodiments, the images are color images. In some embodiments, the images are fluorescent images. In some embodiments, the tissue or cells or the tissue are stained. In some embodiments, the tissue or cells of the tissue are labeled. In some embodiments, the stain and/or label is a fluorescent stain and/or label. In some embodiments, the component is a stained component. A skilled artisan will be familiar with methods of labeling and identifying cells in tissue. Cell type surface markers as well as intracellular markers can be bound by labeled antibodies and/or labeled nucleic acid molecules to identify each cell/cell type. Examples of such tagging/labeling include for example CD20 as a marker for B cells, CD3 as a marker for T cells, CD8 as a marker for CD8+ T cells, CD4 as a marker for CD4+ T cells, CD57 for natural killer (NK) cells, CD14 for macrophages, and CD33 for myeloid derived suppressor cells (MDSCs) among many others. Any known identifying marker may be employed. In some embodiments, the markers are selectedfromthe markers provided inTable 1. In some embodiments, the markers are allmarkers provided in Table 1. In some embodiments, the components are selected from the proteins/genes provided in Table 1. In some embodiments, the components are all the proteins/genes provided in Table 1. In some embodiments, the marker is unique to a target cell or cell/type. In some embodiments, a marker is a component. In some embodiments, a cell type is a component. In some embodiments, the component is a cell-specific components. In some embodiments, the component identifies a cell type of interest. In some embodiments, the component identifies a diseased cell. In some embodiments, each component comprises a unique stain. In some embodiments, each component is uniquely stained. In some embodiments, uniquely stained comprises associated with a uniquely detectable moiety. Detectable moieties are well known in the art and include, for non-limiting example, a fluorophore (e.g., GFP, RFP, YFP, luciferase and the like), a radioactive tag, and a colored tag). Any such known tag may be employed. In some embodiments, associated comprises bound to.

TABLE 1
Markers/components

| Marker | | |
|---|---|---|
| Name | Marker family | Type |
| BCL2 | Functional Markers | Multifunctional |
| Beta-Catenin | Functional Markers | Multifunctional |
| CCR4 | Functional Markers | Cytokine Signaling |
| CD11b | Immune Markers | Macrophages |
| CD11c | Immune Markers | Dendritic Cells |
| CD138 | Immune Markers | Plasma Cells |
| CD15 | Granulocytes | Immune Markers |
| CD163 | Immune Markers | Macrophages |
| CD2 | Immune Markers | T-cells |
| CD20 | Immune Markers | Plasma Cells |
| CD21 | Immune Markers | Dendritic Cells |
| CD25 | Functional Markers | Cytokine Signaling |
| CD3 | Immune Markers | T-cells |
| CD30 | Functional Markers | Activation/Proliferation |
| CD31 | Auxiliary Markers | Blood vessels, lymphatics |
| | | and nerves |
| CD34 | Auxiliary Markers | Blood vessels, lymphatics |
| | | and nerves |
| CD37 | Functional Markers | Multifunctional |
| CD38 | Immune Markers | Plasma Cells |
| CD4 | Immune Markers | T-cells |
| CD44 | Auxiliary Markers | Membrane |
| CD45 | Auxiliary Markers | Membrane |
| CD45RA | Functional Markers | Multifunctional |
| CD45RO | Functional Markers | Multifunctional |
| CD5 | Immune Markers | T-cells |
| CD56 | Immune Markers | NK Cells |
| CD57 | Immune Markers | NK Cells |
| CD68 | Immune Markers | Macrophages |
| CD7 | Immune Markers | T-cells |
| CD71 | Functional Markers | Multifunctional |
| CD8 | Immune Markers | T-cells |
| CDX2 | Auxiliary Markers | Epithelia |
| Chromogranin | Auxiliary Markers | Neuroendocrine |
| Collagen IV | Auxiliary Markers | Extracellular Matrix |
| Cytokeratin | Auxiliary Markers | Epithelia |
| EGFR | Functional Markers | Cytokine Signaling |
| FOXP3 | Immune Markers | T-cells |
| GATA-3 | Immune Markers | T-cells |
| GFAP | Auxiliary Markers | Blood vessels, lymphatics |
| | | and nerves |
| Granzyme B | Functional Markers | Activation/Proliferation |
| HLA-DR | Functional Markers | Multifunctional |
| ICOS | Functional Markers | Activation/Proliferation |
| IDO-1 | Functional Markers | Multifunctional |
| Ki67 | Functional Markers | Activation/Proliferation |
| LAG3 | Functional Markers | Checkpoint |
| MMP12 | Functional Markers | Activation/Proliferation |
| MMP9 | Functional Markers | Activation/Proliferation |
| MUC-1 | Auxiliary Markers | Epithelia |
| Na-K-ATPase | Auxiliary Markers | Membrane |
| p53 | Functional Markers | Multifunctional |
| PD-1 | Functional Markers | Checkpoint |
| PD-L1 | Functional Markers | Checkpoint |
| Podoplanin | AUxiliary Markers | Blood vessels, lymphatics |
| | | and nerves |
| Synaptophysin | Auxiliary Markers | Neuroendocrine |
| T-bet | Immune Markers | T-cells |
| Vimentin | Auxiliary Markers | Cytoplasm |
| Vista | Functional Markers | Checkpoint |
| αSMA | Auxiliary Markers | Smooth Muscle |
| Hoechst | Auxiliary Markers | Nucleus |
| DRAQ5 | | |

In some embodiments, the first state is selected from: a healthy state, a disease state, a state of responding to therapy, a state of non-response to therapy, a state of disease regression, a state of disease stability, a state of disease regression or stability, a state of disease progression, a state of positive disease prognosis, a state of negative disease prognosis, a state of disease resistance and a state of disease susceptibility. In some embodiments, the second state is selected from: a healthy state, a disease state, a state of responding to therapy, a state of non-response to therapy, a state of disease regression, a state of disease stability, a state of disease regression or stability, a state of disease progression, a state of positive disease prognosis, a state of negative disease prognosis, a state of disease resistance and a state of disease susceptibility. In some embodiments, the first state and the second state are combined selected from: a healthy state and a disease state, a state of responding to therapy and a state of non-response to therapy, a state of disease regression or stability and a state of disease progression, a state of positive disease prognosis and a state of negative disease prognosis and a state of disease resistance and a state of disease susceptibility. In some embodiments, the first state and the second state are a healthy state and a disease state. In some embodiments, the first state and the second state are a state of responding to therapy and a state of non-response to therapy. In some embodiments, the first state and the second state are a state of disease regression or stability and a state of disease progression. In some embodiments, the first state and the second state are a state of positive disease prognosis and a state of negative disease prognosis. In some embodiments, the first state and the second state are a state of disease resistance and a state of disease susceptibility.

In some embodiments, a first state is a desired state and the second state is an undesired state. In some embodiments, subjects in a first state are healthy controls. In some embodiments, subjects in a second state are subjects suffering from disease. In some embodiments, subjects in a first state are responders to a treatment. In some embodiments, subjects in a second state are non-responders to a treatment. In some embodiments, subjects in a first state are subjects with stable or regressing disease. In some embodiments, subject in a second state are subjects with progressing disease. In some embodiments, subjects in a first state are subjects with a positive prognosis. In some embodiments, subject in a second state are subjects with a negative prognosis. In some embodiments, subjects in a first state are subjects that are resistant. In some embodiments, subject in a second state are subjects that are susceptible. In some embodiments, resistant/susceptible is to disease. In some embodiments, resistant/susceptible is to treatment.

In some embodiments, a non-responder is a subject that is not responsive to therapy. In some embodiments, a non-responder is a subject with a non-favorable response to therapy. As used herein a “non-favorable response” of the patient indicates “non-responsiveness” of the patient to the treatment and thus the treatment of the non-responsive cancer patient with the therapy will not lead to the desired clinical outcome, and potentially to a non-desired outcomes such as the case of cancer tumor expansion, recurrence and metastases.

In some embodiments, a responder is a subject that is responsive to therapy. In some embodiments, a responder is a subject with a favorable response to therapy. As used herein, a “favorable response” of the patient indicates “responsiveness” of the patient to the treatment with the therapy, namely, the treatment of the responsive patient with the therapy will lead to the desired clinical outcome such as in the case of cancer tumor regression, tumor shrinkage or tumor necrosis; an anti-tumor response by the immune system; preventing or delaying tumor recurrence, tumor growth or tumor metastasis.

In some embodiments, tissue is diseased tissue. In some embodiments, tissue comprises diseased cells. In some embodiments, the image comprises diseased cells. In some embodiments, the tissue is healthy tissue. In some embodiments, the tissue is devoid of diseased cells. In some embodiments, the tissue comprises immune cells. In some embodiments, the immune cells are tissue resident immune cells. In some embodiments, the tissue comprises healthy and diseased tissue. In some embodiments, the image comprises healthy tissue. In some embodiments, the image comprises healthy cells. In some embodiments, the image comprises healthy and diseased tissue/cells. In some embodiments, the healthy cells are of the same cell type as the diseased cells. In some embodiments, the healthy cells are of the same tissue as the diseased cells. In some embodiments, the healthy tissue is the same type of tissue as the diseased tissue.

In some embodiments, the tissue is a therapy treated tissue. In some embodiments, the tissue is from a subject that has received therapy. In some embodiments, the tissue is from a subject that is receiving therapy. In some embodiments, therapy is a drug. In some embodiments, the subject has a known response to the therapy. In some embodiments, the subject has an unknown response to the therapy. In some embodiments, response is selected from being a responder and being a non-responder.

In some embodiments, the ML model is trained. In some embodiments, the ML model is trained to distinguish between tissue samples. In some embodiments, distinguishing is between samples from subjects in a first state and samples from subjects in a second state. In some embodiments, the ML model is trained to assign tissue samples as having originated from subjects in a first state or a second state. In some embodiments, the ML model is trained to diagnose a first state or a second state. In some embodiments, the ML model is trained to predict a subject as being in a first state or a second state. In some embodiments, the subject is the subject that provided the tissue sample. In some embodiments, the tissue sample is the tissue sample that appears in the one or more images. In some embodiments, the subject is the subject that provided the one or more images.

Machine learning models are well known in the art and any such model may be used. Models include, but are not limited to artificial neural networks, support vector machines (SVM) classifier and a k-nearest neighbor (k-NN) classifier. In some embodiments, the machine learning model is a classifier. In some embodiments, the machine learning model is an SVM classifier. In some embodiments, the machine learning model is a k-NN classifier. In some embodiments, the machine learning model is selected from an SVM classifier and a k-NN classifier. In some embodiments, the algorithm is the boost algorithm. In some embodiments, the ML model employs the boost algorithm. In some embodiments, the ML model is the boost algorithm. In Examples of a boost algorithm include for an XGBoost algorithm. In some embodiments, the machine learning model implements a machine learning algorithm. In some embodiments, the machine learning model is a supervised model. In some embodiments, supervised is self-supervised.

In some embodiments, the method further comprises an inference stage. In some embodiments, the inference stage comprises applying the trained ML model. In some embodiments, the trained ML model is applied to the at least one images. In some embodiments, the inference stage comprises predicting the subject state of the subject that provided the images. In some embodiments, the classifying is the inference stage. In some embodiments, the applying the trained ML model is the inference stage.

In some embodiments, the trained ML model is produced by a method comprising receiving one or more extracted spatial features. In some embodiments, the spatial features are extracted from a first set of images. In some embodiments, the first set of images is of tissue from subjects in a first state. In some embodiments, tissue is a tissue sample. In some embodiments, the spatial features are extracted from a second set of images. In some embodiments, the second set of images is of tissue from subject in a second state. In some embodiments, a set is at least 2, 3, 4, 5, 6, 7, 8, 9, 10, 15, 20, 25, 30, 35, 40, 45, 50, 60, 70, 75, 80, 90 or 100 images. Each possibility represents a separate embodiment of the invention. In some embodiments, a set is at least 2 images.

In some embodiments, the images contain spatial positions of components. In some embodiments, the images contain spatial positions of at least two components. In some embodiments, the images contain spatial positions of all components. In some embodiments, all components are all detectable components. In some embodiments, all components are all labeled components. In some embodiments, the images are the set of images. In some embodiments, all images of the set contain the same components. In some embodiments, all images of the set contain the same type of tissue. In some embodiments, the type of tissue in the first set of images is the same type of tissue as in the second set of images. In some embodiments, the components made identifiable in the first set of tissues is the same as the components made identifiable in the second set of images. It will be understood by a skilled artisan that the components examined are the same for both sets, however, it may be that a component is only visible/detectable in one set. For example, a tumor specific antigen may not be present in a set of images from a healthy control. In some embodiments, the components are only components present in both sets of images. In some embodiments, the components are only components present in both the first state and the second state.

In some embodiments, the method further comprises receiving a first set of images from a subject in a first state. In some embodiments, the first set of images is from a plurality of subjects. In some embodiments, the method further comprises receiving a second set of images from a subject in a second state. In some embodiments, the second set of images is from a plurality of subjects. In some embodiments, the subjects from the first set and the subjects from the second set are a plurality of subjects. In some embodiments, a plurality is at least 2, 3, 4, 5, 6, 7, 8, 9, 10, 15, 20, 25, 30, 35, 40, 45, 50, 60, 70, 75, 80, 90, 100, 150, 200, 250, 300, 350, 400, 450, 500, 1000, 2000, 5000, 10000, 50000, or 100000 subjects. Each possibility represents a separate embodiment of the invention. In some embodiments, a plurality is at least 15 subjects. In some embodiments, a plurality is at least 20 subjects.

In some embodiments, the one or more images are from a test subject. In some embodiments, a test subject is a subject with an unknown state. In some embodiments, the one or more images are from a training set. In some embodiments, the training set is the training set upon which the ML model was trained. In some embodiments, the one or more images if from a subject in the first state. In some embodiments, the one or more images if from a subject in the second state.

In some embodiments, the images of the set contain spatial positions. In some embodiments, the spatial positions are of at least two components. In some embodiments, the method further comprises extracting at least one spatial feature from the received one or more images. In some embodiments, the method further comprises extracting at least one spatial feature from each image. In some embodiments, the method further comprises extracting at least one spatial feature from the set of images. In some embodiments, the spatial feature is a spatial relationship. In some embodiments, the relationship is between at least two components. In some embodiments, extracting one or more spatial features comprises assembling a table of spatial features. In some embodiments, extracting one or more spatial features comprises assembling a table of the measure of each spatial feature. In some embodiments, the table comprises all the features and measures from an image. In some embodiments, the table comprises features and measures from each image. In some embodiments, the table comprises features and measures from all images.

In some embodiments, the spatial relationship is a distance. In some embodiments, the spatial relationship is a measure of distance. In some embodiments, distance is at least one distance between at least two components. In some embodiments, the at least one distance is the shortest distance. In some embodiments, the at least one distance is the longest distance. In some embodiments, the spatial relationship is contact. In some embodiments, contact is contact of the at least two components. In some embodiments, the spatial relationship is association. In some embodiments, association is association of the at least two components. In some embodiments, association comprise proximity. In some embodiments, proximity comprises a distance of less than 1000, 500 100, 90, 80, 75, 70, 60, 50, 45, 40, 35, 30, 25, 20, 15, 12, 10 or 5 microns. In some embodiments, the spatial relationship is overlap. In some embodiments, overlap is overlap of the at least two components. In some embodiments, the spatial relationship is structure. In some embodiments, structure is structure of the at least two components. In some embodiments, the spatial relationship is density. In some embodiments, density is density of a component. In some embodiments, density is relative density. In some embodiments, the spatial relationship is abundance. In some embodiments, abundance is abundance of a component. In some embodiments, abundance is relative abundance.

In some embodiments, the extracting comprises measuring distance. In some embodiments, measuring is calculating. In some embodiments, the distance is between the components. In some embodiments, the distance is average distance between the components. In some embodiments, the distance is mean distance between the components. In some embodiments, the distance is the distribution of the components. In some embodiments, the distance is the distribution between the components. It will be understood by a skilled artisan that there may be many copies of a component, for example is the component is CD3, there will be many CD3 proteins molecules on a given T cell and many T cells. Therefore, there will be many CD3 molecules and not just one. This is likely to be true of the second component and so the measurement may not just be a simple linear measure, but rather an average, mean, or distribution that describes the spatial relationship between the two components. In some embodiments, the extracting comprises measuring relative position. In some embodiments, the measure is an absolute measure. In some embodiments, the measure is a relative measure. In some embodiments, the measure is a 3-dimensional measure. In some embodiments, the distance is a magnitude. In some embodiments, the distance is a vector.

In some embodiments, the method of producing a trained ML model comprises generating training set. In some embodiments, the training set is an annotated training set. In some embodiments, the training set comprises the extracted spatial relationships. In some embodiments, the training set comprises labels. In some embodiments, the labels correspond to the extracted spatial relationships. In some embodiments, the labels indicate the origin of the spatial relationship. In some embodiments, the origin is wherefrom the spatial relationship was extracted. In some embodiments, the origin is from an image from a subject in a first state or a subject in second state. In some embodiments, the label is the first state or the second state. In some embodiments, the label is from a subject in the first state or a subject in the second state.

In some embodiments, the method of producing a trained ML model comprises training the ML model. In some embodiments, the training is training the algorithm. In some embodiments, the training is training on the training set. In some embodiments, the training comprises providing the training set to the model. In some embodiments, the training comprises running the model on the training set. In some embodiments, the training produces a trained ML model. In some embodiments, the training produces a model capable of distinguishing between images from subject in the first state and images from subject in the second state. In some embodiments, images from a subject are images of a tissue from a subject.

In some embodiments, the extracting is by an ML model. In some embodiments, the extracting is by an interpretability model. In some embodiments, the interpretability model is an ML model. In some embodiments, the interpretability model outputs pairs of biological components. In some embodiments, the pairs are ones whose spatial relationship contributed to the classifying. In some embodiments, contributed is significantly contributed. In some embodiments, significantly is statistically significantly. In some embodiments, a list of pairs is output. In some embodiments, the pairs are ordered by the magnitude of contribution. In some embodiments, the list is ordered by magnitude. In some embodiments, the list is ordered by ability to contribute. In some embodiments, the pairs are ordered by the ability to contribute. In some embodiments, magnitude is magnitude of contribution. In some embodiments, magnitude is magnitude of significance. In some embodiments, the most highly contributing are selected. In some embodiments, the most highly comprises the 1, 2, 3, 4, 5, 6, 7, 8, 9, 10, 12, 15, 17, 20, 25, 30, 35, 40, 45, 50 or 100 most contributing. Each possibility represents a separate embodiment of the invention. In some embodiments, the list comprises at least 1, 2, 3, 4, 5, 6, 7, 8, 9, 10, 12, 15, 17, 20, 25, 30, 35, 40, 45, 50 or 100 pairs. Each possibility represents a separate embodiment of the invention. It will be understood by a skilled artisan that the contribution will change depending on the image or set of images being analyzed by the ML model. Thus, the ML model may be run on multiple images or sets of images in order to obtain more target pairs. In some embodiments, the ML model is applied until no new target pairs are output.

In some embodiments, the method further comprises designing a drug that alters a spatial relationship between the pair of components. In some embodiments, the drug is a small molecule. In some embodiments, the drug is a biologic. In some embodiments, the drug is an antibody or antigen binding fragment thereof. In some embodiments, the antibody or antigen binding fragment is multi-specific. In some embodiments, multi-specific is bi-specific. In some embodiments, multi-specific is specific to the at least two components. In some embodiments, bispecific is specific to the pair of components. In some embodiments, the drug is bispecific. In some embodiments, the drug binds at least one of the components. In some embodiments, the drug binds both of the components. In some embodiments, specific to is binds to. In some embodiments, specific to is binds exclusively to. In some embodiments, specific to is does not significantly bind to another target.

In some embodiments, the pair of components are closer to each other in the second state than the first state. In some embodiments, the pair of components are closer to each other in the first state than the second state. In some embodiments, the pair of components are farther away from each other in the second state than the first state. In some embodiments, the pair of components are farther away from each other in the first state than the second state. In some embodiments, the first state is a desired state, and the second state is an undesired state and the pair of components are closer to each other in the second state than the first and the drug distances the pair of components. In some embodiments, distancing comprises disrupting association. In some embodiments, association is binding. In some embodiments, distancing comprises sequestering at least one of the components. In some embodiments, distancing comprises blocking association. In some embodiments, blocking is inhibiting. In some embodiments, the drug is a blocking antibody. In some embodiments, the drug binds one of the components. In some embodiments, the first state is a desired state, and the second state is an undesired state and the pair of components are closer to each other in the first state than the second state and the drug draws the two components nearer. In some embodiments, nearer is nearer to each other. In some embodiments, distances is distances from each other. In some embodiments, drawing nearer comprises bringing into contact. In some embodiments, drawing nearer comprises causing association. In some embodiments, drawing nearing comprises brining into proximity. In some embodiments, the drug is bispecific for the two components. In some embodiments, drawing nearing comprises binding at least one of the components and bringing it to a site thereby increasing its abundance and/or density at the site. In some embodiments, the site is a disease site. In some embodiments, the disease site is a tumor. In some embodiments, the disease site is the tumor microenvironment (TME). In some embodiments, at least one component is one of the components. In some embodiments, at least one component is both of the components.

In some embodiments, the method further comprises administering the designed drug to a subject. In some embodiments, the subject is a subject in need thereof. In some embodiments, the subject in a subject in the second state. In some embodiments, the subject is a subject in the undesired state. In some embodiments, the subject is a mammal. In some embodiments, the subject is a human. In some embodiments, the subject needs a method of the invention. In some embodiments, the subject needs treatment. In some embodiments, the drug is for use in treating the undesired state. In some embodiments, the drug is for use in converting a subject in the undesired state to the desired state.

By another aspect, there is provided a method of generating an agent, the method comprising identifying at least two components by a method of the invention wherein the at least two components are closer to each other in the second state that the first state, and designing a drug that distances the components, thereby generating an agent.

By another aspect, there is provided a method of generating an agent, the method comprising identifying at least two components by a method of the invention wherein the at least two components are closer to each other in the first state that the second state, and designing a drug that draws nearer the components, thereby generating an agent.

By another aspect, there is provided a method of converting a subject in a second state into a subject in a first state, the method comprising administering to the subject a drug that distances a pair of components, wherein the pair is selected by a method of the invention, thereby converting a subject.

By another aspect, there is provided a method of converting a subject in a second state into a subject in a first state, the method comprising administering to the subject a drug that draws together a pair of components, wherein the pair is selected by a method of the invention, thereby converting a subject.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random-access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

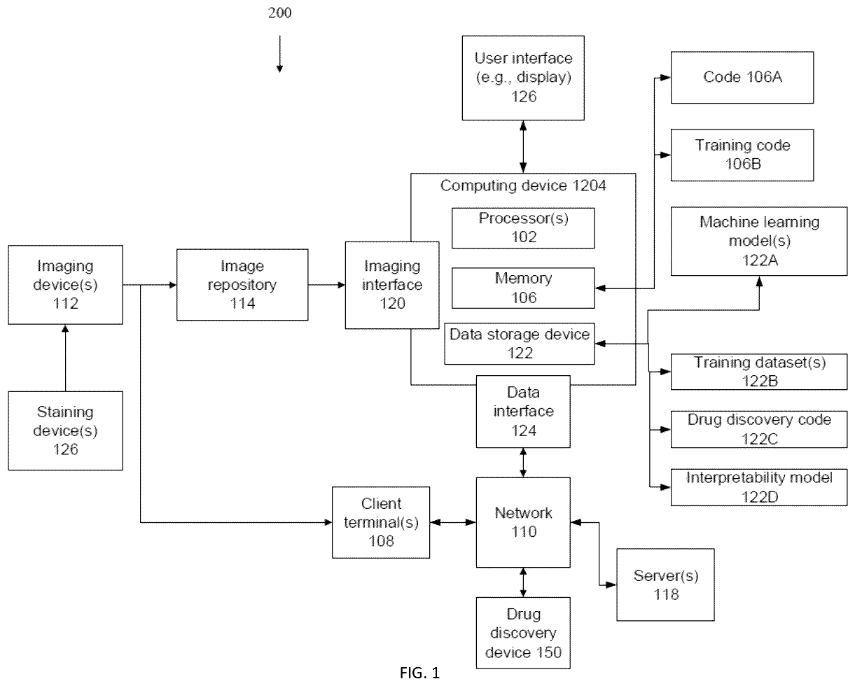

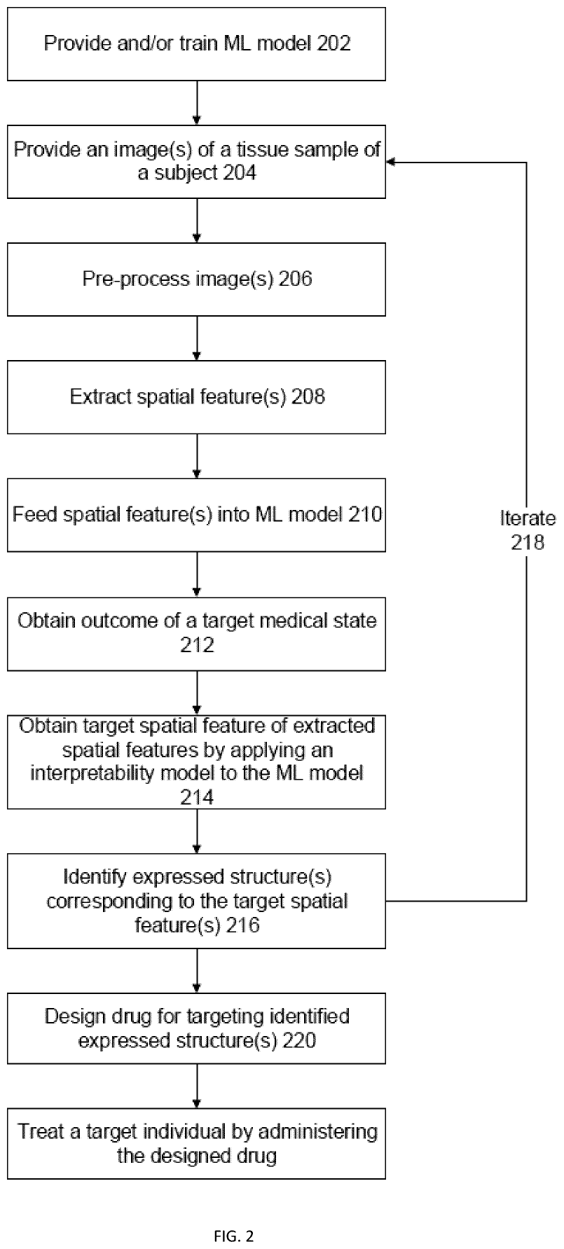

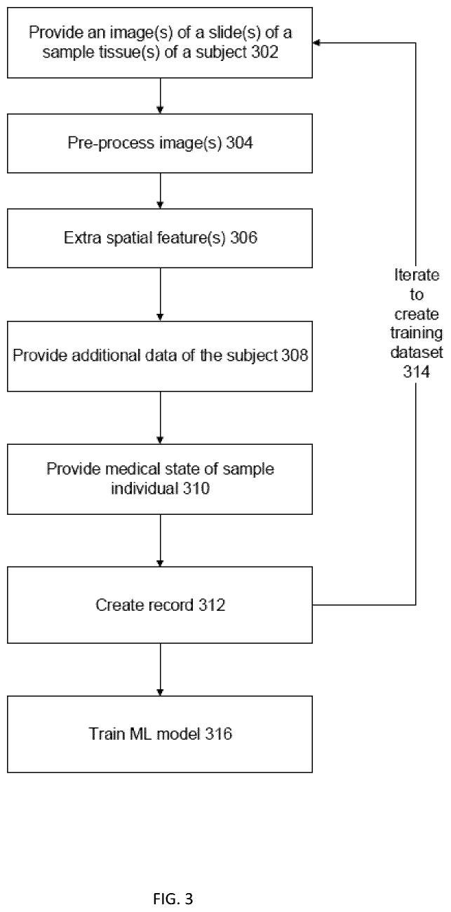

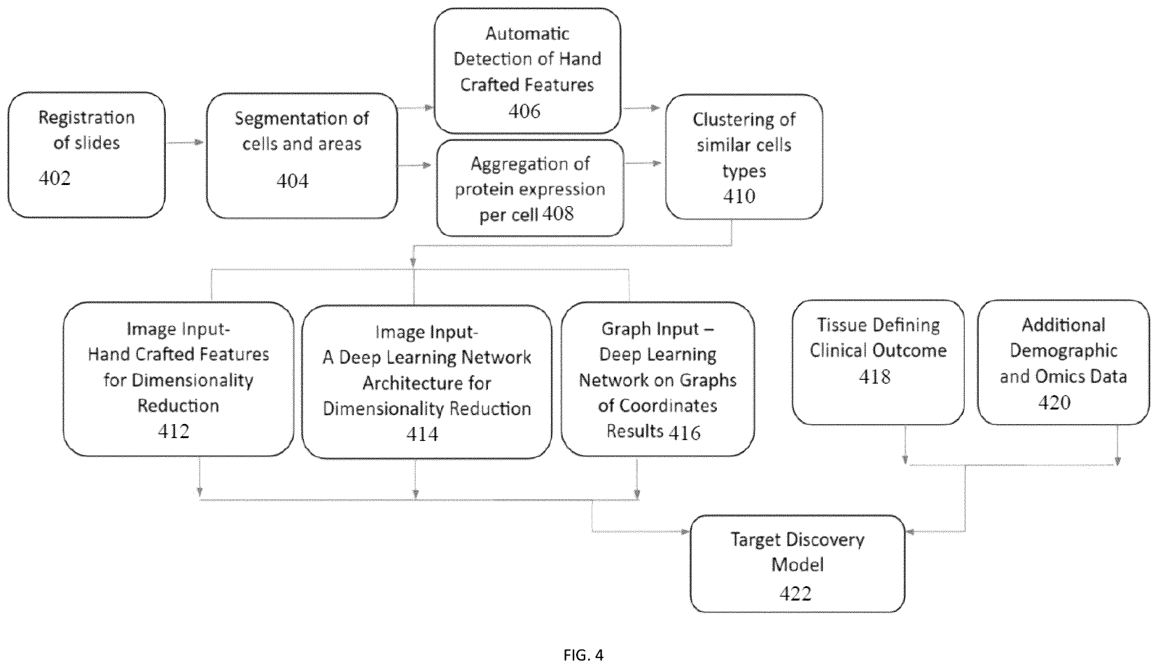

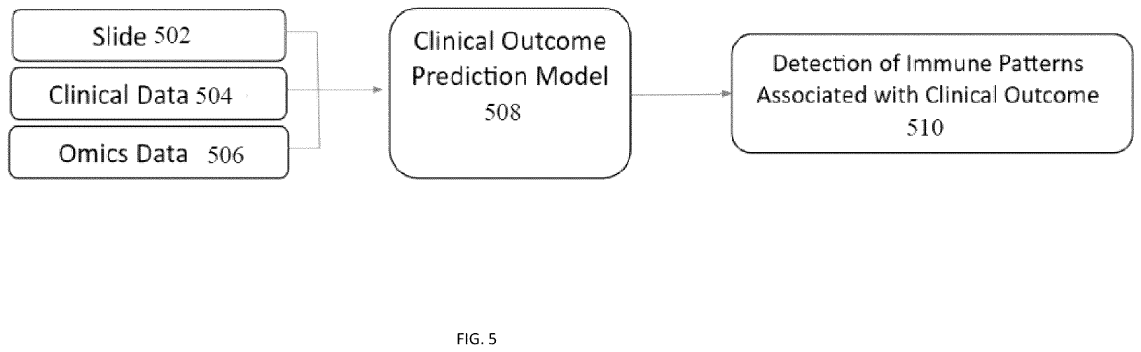

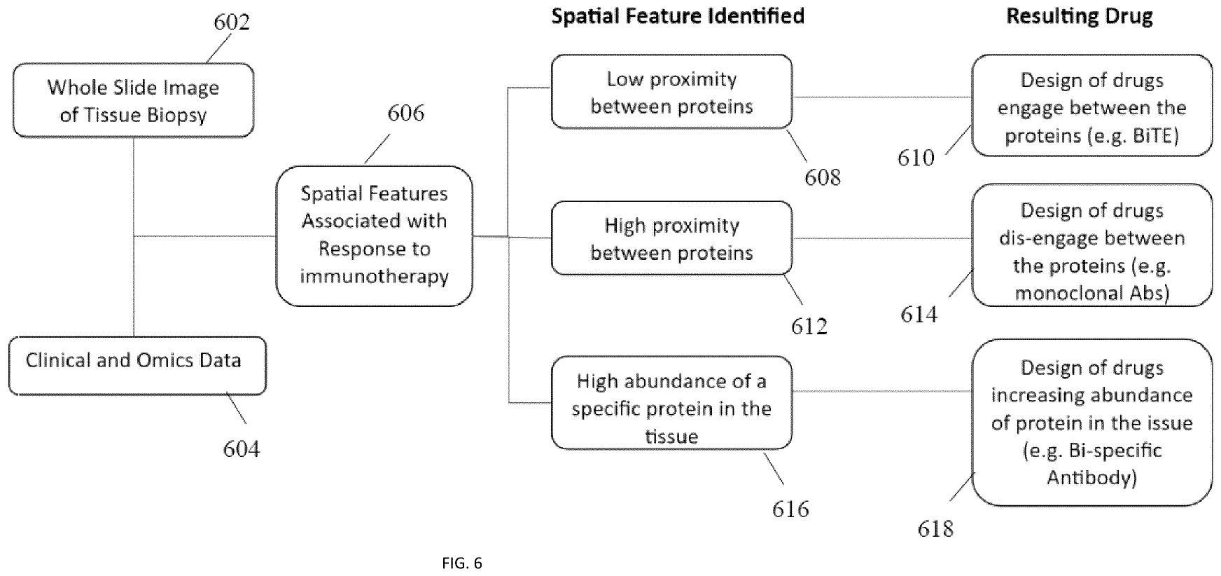

Reference is now made to FIG. 1, which is a block diagram of a system 100 for identifying two or more expressed structures for developing a drug for treating subjects suffering from a medical condition, in accordance with some embodiments of the present invention. Reference is also made to FIG. 2, which is a flowchart of a method of identifying two or more expressed structures for developing a drug for treating subjects suffering from a medical condition, in accordance with some embodiments of the present invention. Reference is also made to FIG. 3, which is a flowchart of a method for training a machine learning model that generates an outcome of a target medical state of a person in response to an input of spatial features, in accordance with some embodiments of the present invention. Reference is also made to FIG. 4, which is a flowchart of another exemplary approach for identifying two or more expressed structures for developing a drug for treating subjects suffering from a medical condition, in accordance with some embodiments of the present invention. Reference is also made to FIG. 5, which is a flowchart of an exemplary high level process for identifying two or more expressed structures for developing a drug for treating subjects suffering from a medical condition, in accordance with some embodiments of the present invention. Reference is also made to FIG. 6, which is a flowchart of an exemplary process for developing drugs for treating subjects suffering from a medical condition, in accordance with some embodiments of the present invention.

System 100 may implement the acts of the method described with reference to FIGS. 2-6, optionally by a hardware processor(s) 102 of a computing device 104 executing code instructions 106A and/or 106B stored in a memory 106.

At least some of the systems and/or methods described herein may implemented and/or integrated features and/or components described with reference to PCT Patent Application Publication No. WO2019/026081 “SYSTEMS AND METHODS FOR ANALYSIS OF TISSUE IMAGES”, and/or as described with reference to PCT Patent Application Publication No. WO2021/001831 “SYSTEMS AND METHODS FOR SELECTING A THERAPY FOR TREATING A MEDICAL CONDITION OF A PERSON” by at least one common inventor of the present disclosure, incorporated herein by reference in its entirety.

Computing device 104 may be implemented as, for example, a client terminal, a server, a virtual server, a laboratory workstation (e.g., pathology workstation), a procedure (e.g., operating) room computer and/or server, a drug discovery station, a virtual machine, a computing cloud, a mobile device, a desktop computer, a thin client, a Smartphone, a Tablet computer, a laptop computer, a wearable computer, glasses computer, and a watch computer. Computing 104 may include an advanced visualization workstation that sometimes is implemented as an add-on to a laboratory workstation and/or other devices for presenting expressed proteins and/or cell types corresponding to identified target spatial features for drug discovery and/or development and/or other computer added detections to the user (e.g., pathologist, oncologist).

Computing device 104 may include locally stored software that performs one or more of the acts described with reference to FIGS. 2-6, and/or may act as one or more servers (e.g., network server, web server, a computing cloud, virtual server) that provides services (e.g., one or more of the acts described with reference to FIGS. 2-6) to one or more client terminals 108 (e.g., remotely located laboratory workstations, remotely located drug discovery terminals, remote picture archiving and communication system (PACS) server, remote electronic medical record (EMR) server, remote tissue image storage server, remotely located pathology computing device, client terminal of a user such as a desktop computer) over a network 110, for example, providing software as a service (SaaS) to the client terminal(s) 108, providing an application for local download to the client terminal(s) 108, as an add-on to a web browser and/or a tissue imaging viewer application, and/or providing functions using a remote access session to the client terminals 108, such as through a web browser. In one implementation, multiple client terminals 108 each obtain images of slides of tissue from different imaging device(s) 112. Each of the multiple client terminals 108 provides the images to computing device 104, and receives back a respective indication of expressed proteins and/or cell types corresponding to identified target spatial features for drug discovery and/or development. In another implementation, code 106A and/ML model(s) 122A are implemented by computing device 104 which receives tissue images from imaging device 112, and provides the indication of expressed proteins and/or cell types corresponding to identified target spatial features for drug discovery and/or development, for example, for presentation on a display (e.g., 126), and/or for input into a drug discovery process 122C (e.g., code, application), and/or for input into a drug discovery device 150. Is it noted that the training of the ML model(s) 122A, and the analysis of tissue images by the trained ML model(s) 122A, may be implemented by the same computing device 104, and/or by different computing devices 104, for example, one computing device trains the ML model(s) 122A, and transmits the trained ML model(s) 122A to a server device for analysis of tissue images to identify expressed proteins and/or cell types corresponding to identified target spatial features for drug discovery and/or development.

Computing device 104 receives tissue images captured by one or more imaging device(s) 112. Exemplary imaging device(s) 112 include: a scanner scanning in standard color channels (e.g., red, green blue), a multispectral imager acquiring images in four or more channels, a confocal microscope, and/or other imaging devices as described herein, a black a white imaging device, an imaging sensor. Multiple images may be acquired for the same slide, for example, depicting different biomarker stains.

Imaging device(s) 112 creates tissue images from physical tissue samples which may be obtained by a tissue extracting device, for example, a fine needle for performing fine needle aspiration (FNA), a larger bore needle for performing a core biopsy, and a cutting tool (e.g., knife, scissors, scoop) for cutting out a sample of the tissue (e.g., tumor removal).

Imaging device(s) 112 may create two and/or three (2D and/or 3D) dimensional tissue images.

Tissue images captured by imaging machine 112 may be stored in an image repository 114, for example, a storage server, a computing cloud, virtual memory, and a hard disk. Training images 116 may be created based on the captured tissue images, by labelling the images with ground truth labels (e.g., obtained from health records of the subject, such as from server(s) 118), for example, as described herein.

Training dataset 122B may be created from training images 116 and other data, including other additional personal data of the subject such as medical history, omics data, and/or demographic data, as described herein. Training dataset 122B is used to train ML model(s) 122A, as described herein.

Drug discovery code 122C (e.g., application) and/or drug discovery device 150 (e.g., drug synthesizer) may be used to design and/or create drugs using the expressed proteins and/or cell types corresponding to identified target spatial features, as described herein.

It is noted that training images 116 may be stored by a server 118, accessibly by computing device 104 over network 110, for example, a publicly available training dataset, tissue images stored in a PACS server and/or pathology imaging server, and/or a customized training dataset created for training the ML models, as described herein.

Computing device 104 may receive the training images 116 and/or tissue images for analysis from imaging device 112 and/or image repository 114 using one or more imaging interfaces 120, for example, a wire connection (e.g., physical port), a wireless connection (e.g., antenna), a local bus, a port for connection of a data storage device, a network interface card, other physical interface implementations, and/or virtual interfaces (e.g., software interface, virtual private network (VPN) connection, application programming interface (API), software development kit (SDK)). Alternatively or additionally, Computing device 104 may receive the training images 116 and/or tissue images for analysis from client terminal(s) 108 and/or server(s) 118.

Hardware processor(s) 102 may be implemented, for example, as a central processing unit(s) (CPU), a graphics processing unit(s) (GPU), field programmable gate array(s) (FPGA), digital signal processor(s) (DSP), and application specific integrated circuit(s) (ASIC). Processor(s) 102 may include one or more processors (homogenous or heterogeneous), which may be arranged for parallel processing, as clusters and/or as one or more multi core processing units.

Memory 106 (also referred to herein as a program store, and/or data storage device) stores code instruction for execution by hardware processor(s) 102, for example, a random access memory (RAM), read-only memory (ROM), and/or a storage device, for example, non-volatile memory, magnetic media, semiconductor memory devices, hard drive, removable storage, and optical media (e.g., DVD, CD-ROM). Memory 106 stores code 106A that implements one or more acts and/or features of the method described with reference to FIGS. 2-6, and/or training code 106B that executes one or more acts of the method described with reference to FIG. 3.

Computing device 104 may include a data storage device 122 for storing data, for example, ML model(s) 122A, training dataset 122B, drug discovery code 122C, interpretability model 122D, and/or other code such as for segmenting of cells and/or extraction of features (e.g., neural networks and/or other classifiers), as described herein. Data storage device 122 may be implemented as, for example, a memory, a local hard-drive, a removable storage device, an optical disk, a storage device, and/or as a remote server and/or computing cloud (e.g., accessed over network 110). It is noted that code portions of the data stored in data storage device 122 may be loaded into memory 106 for execution by processor(s) 102.

Computing device 104 may include data interface 124, optionally a network interface, for connecting to network 110, for example, one or more of, a network interface card, a wireless interface to connect to a wireless network, a physical interface for connecting to a cable for network connectivity, a virtual interface implemented in software, network communication software providing higher layers of network connectivity, and/or other implementations. Computing device 104 may access one or more remote servers 118 using network 110, for example, to download updated training images 116 and/or to download an updated version of the ML model(s) 122A, training code 106B, and/or the training dataset 122B.

Computing device 104 may communicate using network 110 (or another communication channel, such as through a direct link (e.g., cable, wireless) and/or indirect link (e.g., via an intermediary computing device such as a server, and/or via a storage device) with one or more of:
Client terminal(s) 108, for example, when computing device 104 acts as a server providing services (e.g., SaaS) to remote laboratory terminals, for identifying expressed proteins and/or cell types corresponding to identified target spatial features for drug discovery and/or development.
Server 118, for example, implemented in association with a PACS and/or electronic medical record, which may storage large numbers of tissue images for analysis and/or which may store personal data of the subject (i.e., subject data) 118A which is analyzed to identify spatial features, as described herein.
Tissue image repository 114 that stores training images 116 and/or tissue images outputted by imaging device 112.
Drug discovery device 150 that discovers and/or synthesizes new immuno-oncology drugs based on expressed proteins and/or cell types corresponding to identified target spatial features.

It is noted that imaging interface 120 and data interface 124 may exist as two independent interfaces (e.g., two network ports), as two virtual interfaces on a common physical interface (e.g., virtual networks on a common network port), and/or integrated into a single interface (e.g., network interface).

Computing device 104 includes or is in communication with a user interface 126 that includes a mechanism designed for a user to enter data (e.g., personal data of the subject) and/or view the expressed proteins and/or cell types corresponding to identified target spatial features for drug discovery and/or development. Exemplary user interfaces 126 include, for example, one or more of, a touchscreen, a display, a keyboard, a mouse, and voice activated software using speakers and microphone.

Referring now back to FIG. 2, at 202, a machine learning (ML) model is trained and/or provided.

Exemplary architectures of the machine learning models described herein include, for example, statistical classifiers and/or other statistical models, neural networks of various architectures (e.g., convolutional, fully connected, deep, encoder-decoder, recurrent, graph), support vector machines (SVM), logistic regression, k-nearest neighbor, decision trees, boosting, random forest, a regressor, and/or a combination of multiple sub-architectures, for example, integrated networks, parallel networks, and/or cascade networks, and/or any other commercial or open source package allowing regression, classification, dimensional reduction, supervised, unsupervised, semi-supervised or reinforcement learning. Machine learning models may be trained using supervised approaches and/or unsupervised approaches. In some implementations, the ML model includes a graph neural network.

The machine learning model is trained on a training dataset of multiple records. Each record includes one or more sample images of one or more sample slides depicting at least a portion of a sample tissue of a sample individual, and a ground truth label indicating a medical state of the sample individual.

The medical state may be selected from a defined group of medical states, where each member of the group represents a specific classification label and/or category. Examples of groups of medical states and classification categories of the groups include, but are not limited to: (i) normal disease versus disease tissue, (ii) response versus non-responder to a certain administered drug, (iii) good prognosis without treatment versus poor prognosis without treatment, (iv) early stage disease versus late stage disease, (v) sensitivity or resistance to an administered immunotherapy pre and/or post treatment.

Records may include the image(s) from which the spatial features are extracted.

Records may include a medical condition of the respective sample individual.

Exemplary medical conditions include, but are not limited to: cancer, optionally a certain type of cancer (e.g., lung cancer, skin cancer, breast cancer, colon cancer), an immune-based medical condition (e.g., autoimmune (e.g., IBD), ophthalmology, infectious, metabolic, hematologic, tissue repair and neurology condition.

Records may include personal data for the respective sample individual. Examples of personal data include: omics data, medical history, clinical outcome, clinical data, and demographic data, and administered treatment (e.g., immunotherapy and/or one or more treatments (e.g., surgical resection, chemotherapy, radiation therapy)).

Optionally, records of the training dataset include multiple slides stained with different stains depicting different structures. The multiple images may be registered with each other.

Optionally, one or more ML models are trained and/or provided. A specific ML model may be selected, for example, according to a target medical condition for which a drug is being designed.

The selection of the ML model may be performed manually by the user (e.g., via a GUI, for example, via a menu and/or icons of available image analysis code). The selection may be performed automatically by code that analyzes, for example, the tissue image, metadata of the image, and/or other patient data associated with the image such as diagnosis of the medical condition and/or proposed treatment type (e.g., obtained from a PACS server, Digital Imaging and Communications in Medicine (DICOM) data, and/or electronic medical record).

At 204, one or more images of one or more tissue samples of a subject, optionally images of pathological slides, are provided (e.g., received and/or accessed).

It is noted that the term ‘slide’ is an exemplary implementation, and other approaches for depicting the sample tissue may be used, for example, live cells in a receptable, a vial, a microarray, and/or a microfluidic chip. Images described herein may refer to images depicting the other captured implementations.

The image(s) of slide(s) depict at least a portion of a target tissue of the person stained with one or more stains indicative of presence of biomarkers in the stained cells, and/or depicting biomarkers in the stained cells (sometimes referred to herein as biomarker stains, or stains).

The term “structures” used herein may sometimes refer to the structures that are stained by the applied stain.

The target tissue may depict the medical condition, for example, cancerous tissue, such as a tumor and/or other malignancy. Additional non-target tissues may be depicted in the images, for example, immune cells, types of different immune cells, and non-target-non-immune cells such as other cells of the body for example red blood cells, blood vessel cells, muscle cells, bone cells, fibroblasts, epithelial cells, and connective tissue cells.

The tissue may be obtained intra-operatively, during for example, a biopsy procedure, a FNA procedure, a core biopsy procedure, colonoscopy for removal of colon polyps, surgery for removal of an unknown mass, surgery for removal of a benign cancer, and/or surgery for removal of a malignant cancer, surgery for treatment of the medical condition. Tissue may be obtained from fluid, for example, urine, synovial fluid, blood, and cerebral spinal fluid.

Tissue may be in the form of a connected group of cells, for example, a histological slide. Tissue may be in the form of individual or clumps of cells suspended within a fluid, for example, a cytological sample.

The images may be obtained, for example, from an image sensor that captures the images, from a scanner that captures images, from a server that stores the images (e.g., PACS server, EMR server, pathology server). For example, tissue images are automatically sent to analysis after capture by the imager and/or once the images are stored after being scanned by the imager.

The images may be whole slide images (WSI). The images may be of slides created from a tissue biopsy obtained from the individual.

Each slide may be stained with one or more biomarker stains, for example, immunohistochemistry (IHC), fluorescence, Hematoxylin and Eosin (H&E), Multiplex Ion Beam Imaging (MIBI), immunofluorescence (IF), Multiplex immunofluorescence (mIF), and the like.

One or more images may be captured for each slide, for example, using different imaging modalities, which may capture different images of the same slide based on different stains. The images for each slide may be multiplexed, for example, stored as multiple channels.

Optionally, the tissue image created from the physical slide with tissue thereon is a color image, optionally including multiple channels for each pixel, for example, 3 (e.g., RGB) or more channels (e.g., multispectral, confocal, fluorescent). Optionally, the tissue image is created based on visible light energy. For example, capturing a digital image of a view as seen under a light microscope. Alternatively or additionally, the tissue image is created based on other radiation wavelengths, for example, near infrared, short wave infrared, and the like.

The tissue may be arranged on a slide. A frozen section may be created and sliced for creating multiple slides. Tissue may be stained. Tissues may be prepared using a formalin-fixed paraffin embedded (FFPE) process.

The slides may include histology slides and/or cytology slides.

The tissue may be chemically stained for increased visibility for generation of the tissue image. Alternatively or additionally, the tissue itself is not stained, but rather imaging methods are used that do not necessarily require staining, for example, a spectral imager.

As used herein, the term biomarker stain sometimes refers to actual staining of the tissue, and/or to ‘virtual staining’ of tissues where the tissue itself is not actually stained but different imaging methods are used that do not necessarily require staining. It is noted that different imaging methods may be combined with different biomarker stains to create multiple combinations of biomarker stains and imaging modalities.

Optionally, a set of colors associated with the chemical staining and/or virtual staining (e.g., by a multispectral imager) is identified. The set of colors may be stored, for example, in a dataset according to the chemical staining and/or virtual staining. The set of colors may be automatically identified by code and/or manually designated by the user according to the chemical and/or virtual staining. The identified set of colors may be used for segmenting tissue versus non-tissue background, and/or for cell type segmentation, as described herein in additional detail. The identified set of colors may be stored, for example, in a LAB color space, RGB color space, and/or other color spaces. It is noted that LAB color space is more linear than RGB color space.

The tissue image may be created by imaging the tissue with the imaging device. Optionally, slides including the prepared tissue are imaged by the imaging device.

Optionally, the tissue slides are imaged at high magnification, for example, between about X200-X400, or about X100-400, or about X100-X200, or about X100, or about X200, or about X400, or other values. Such high magnification imaging may create very large images, for example, on the order of Giga Pixel sizes. Such large tissue images of the entire slide may be referred to herein as Whole Slide Images (WSI).

The imaging device may be implemented as, for example, a spectral imager, such as a multispectral (few to tens of channels) or a hyperspectral (up to hundreds of channels). The multispectral imager creates tissue images with 4 or more spectrum frequencies, which is noted to be higher than the 3 spectrums of a normal imager (e.g., imaging in red, green, and blue (RGB). The imager may produce a spectral signature including multiple channels for each pixel, in contrast for example, to the 3 channels (e.g., RGB) obtained by the traditional staining process. The image analysis code described herein may be created and/or trained according to the spectral signature of each pixel. It is noted that alternatively, a standard imager imaging in 3 channels (e.g., RGB) may be used, and/or a black and white imager may be used.

Alternatively or additionally, the imaging device is implemented based on a Stimulated Raman scattering (SRS) microscopy. The spectral image (cube) acquired by a spectral imager, or an SRS microscope, may be analyzed by combining morphological based method with spectral based methods to improve the outcome of traditional image analysis methods relying purely on RGB images.

Alternatively or additionally, a mapping and/or other transformation function is estimated between the colors (e.g., RGB) of an image of stained tissue and the spectrum corresponding to the same location. The mapping may be used to produce a virtual stained slide from a spectral image of a fresh tissue slide.

Multiple tissue images of the tissue may be provided, for example, from the same biopsy, of different stains, of the same body fluid, a slices from a sequential slicing (e.g., frozen section). The multiple tissue images may be arranged as a single 3D tissue image, and/or as a set of 2D slices. The multi-slide level tissue type(s) may be computed according to an analysis of the multiple tissue images, as described herein.

The slides may be obtained from a volume (i.e., three dimensions (3D)) of tissue by a parallel slicing process, for example, a knife that slices the volume into parallel slices. The slides (which may be processed as two dimensional (2D) images) are created from the parallel slices. The slides may be obtained from different regions of the volume of tissue, which may lie along the same plane. The different slides may correspond to different regions of the tumor, for example, within the tumor, the external boundary of the tumor, and different surfaces of the tumor.

Additional personal data of the subject (whose tissue is depicted in the images) may be obtained, for example, automatically from a dataset (e.g., EMR, PACS server), and/or manually entered (e.g., via a user interface).

Exemplary additional personal data of the subject (where the same type of additional data is included in records used to train the ML model, as described herein) includes, but is not limited to one or more of:
Clinical data of the subject, for example, medical history, demographic data, omics data, age, gender, tumor stage, and the like.
Exemplary omics data includes: genetic mutations, microsatellite instability, and the like.
Administered therapy for treatment of the subject. The administered therapy may be a general modality type of therapy and/or combination thereof, for example, one or combination of: immunotherapy, chemotherapy, and radiation therapy. The administered therapy may be a particular type of therapy and/or combination thereof, for example, a certain combination of chemotherapy drugs, a certain immunotherapy drug, and/or combination of the certain chemotherapy drugs and certain immunotherapy drug.
Type of medical condition, for example, general medical condition, such as cancer. The type of medical condition may be a specific type, such as a sub-type of the general medical condition, for example, type of cancer, such as colon cancer, breast cancer, lung cancer, and skin cancer.

At 206, the image(s) may be pre-processed. One or more exemplary pre-processing features are now described.

The pre-processing may include registration of the images with each other. Registration may be performed between images of slides stained with different stains depicting different structures.

The pre-processing may include dividing the image(s) into patches. Each patch may have a maximal distance (e.g., length, width, diameter) selected according to likelihood of an interaction between two cells located at ends of the maximal distance.

The pre-processing may include creating segmentations, by segmenting the image, into cell type segmentations and/or region type segmentations. For each of the cell type segmentations, cell phenotype features and/or at least one expressed structure may be extracted from an analysis of the stains. The cell type segmentations may be clustered according to a clustering requirement(s) to create clusters. An exemplary clustering requirement includes a requirement that each respective cluster includes only a single respective cell type segmentation and/or that each respective cluster include a unique combination of expressed structures. One or more of the following may be assigned to each respective cell type segmentation and/or each respective cluster: a feature vector including the cell phenotype features, at least one expressed structure extracted for the respective segmentation, and an indication of a location of the cell type segmentation relative to one or more region type segmentations.

Additional details of one or more exemplary and/or optional preprocessing features are now described.

The received images of slices may be registered. Registration may be performed in 2D (e.g., mapping corresponding locations and/or structures in the images to the same 2D location) and/or 3D.

Optionally, images depicting multiple slides obtained from the 3D volume of tissue by the parallel slicing process depicting multiple biomarker stains are registered. The registration may be to create a single 2D image, and/or a registered 3D volume. The segmenting (as described herein) is performed for the 2D image and/or for the 3D volume.

Multiple segmentations may be computed, by segmenting each of the images.

The images may be segmented into multiple structure segmentations, optionally cell type segmentations. Optionally, each segmentation includes a single structure (e.g., single cell) of a certain structure type (e.g., cell type). For example, there may be multiple cell types, for example, immune cells, sub-types of immune cells (e.g., T cells, B cells, lymphocytes, macrophages), platelets, cancer cells, red blood cells, blood vessels, bone cells, fat cells, muscle cells, fibroblasts, epithelial cells, connective tissue cells non-immune-non-cancer cells. Cell type segmentations may include bacteria, protozoa, and/or other non-human cells. Cell type segmentations may include cancer cells and/or cells of the target tissue associated with the medical condition.

Alternatively or additionally, the images are segmented into multiple region type segmentations. The region type segmentation may include multiple cells, which may be of different types, and/or may include tissues (or portion thereof) and/or microenvironments. For example, blood vessels, bone tissue, fat tissue, fibroblasts, epithelial cells, muscle tissue, connective tissue, lymph node, stroma, tumor region, tumor microenvironment, microenvironment of the target tissue associated with the medical condition, and the target tissue associated with the medical condition.

Each cell type and/or region type segmentation (e.g., when registered) and/or other structure segmentation, may corresponding to multiple biomarker stained, for example, each segmentation is registered to multiple images depicting multiple biomarker stains. For example, the same segmented lymphocyte may be depicted in one image with one biomarker stain, and in another image with another biomarker stain.

Optionally, segmentation is performed for the 3D registered volume, i.e., for the images depicting slides obtained from the 3D volume of tissue (e.g., by the parallel slicing process. In such a case, a set of three dimensional (3D) coordinates denoting location of the respective segmentation within the 3D volume may be computed. In such implementation, nodes of the cell-graph (computed as described herein) are associated with the set of 3D coordinates. The physical distance associated with the edges of the graph (as described herein) is computed as the distance within the 3D volume between the 3D coordinates of the respective nodes.

The segmentation may be performed to identify, for example, individual cells, groups of same type of cells, groups of different types of cells, and/or tissues.

The segmentations may be performed by segmentation code, for example, a neural network (e.g., CNN) trained to perform the segmentation using a training dataset of labelled segmentations.

Optionally, each image depicting a respective stain and/or captured by a respective imaging modality may be segmented. When the images are registered, segmentations of the multiple registered images may correspond to the same physical region being segmented, for example, providing a multi-channel segmentation.

Additional exemplary processes for segmenting the images of the slides are described, for example, with reference to WO2019/026081, for example, feature 110 of FIG. 1 of WO2019/026081.

Optionally, for each of the segmentations (e.g., cell type and/or region type and/or other structure), one or more cell phenotype features are extracted. The cell phenotype features are extracted based on an analysis of the biomarker stains. Each cell phenotype feature may be extracted from one respective biomarker stain of the corresponding cell type segmentation, and/or a combination of two or more biomarker stains for the corresponding cell type segmentation.

Exemplary cell phenotype features include, but are not limited to one or a combination of:
An indication of the type of stain(s) (e.g., biomarker stain) and/or capturing method used within the respective segmentation (e.g., PD-L1 IHC, fluorescence, H&E, MIBI)
A size depicted within the segmentation (e.g., area, percentage of area within the segmentation) that includes the biomarker stain, stain intensity (e.g., distribution, histogram of number of pixels and corresponding stain intensity) of one or combination of biomarker stains depicted in the respective segmentation stained.
Stain intensity of one or a combination of biomarkers within each cell within the respective segmentation. The stain intensity may include various intensities such as nuclear or membrane staining intensity. For example—the PD-L1 membrane staining intensity.
An indication of cell morphology of each cell and/or the nucleus of the cell within the respective segmentation, for example, eccentricity of the respective cell.
Area of the respective cell and/or the nucleus of the respective cell (e.g., surface area, and/or volume).
Type and/or classification category of cell, for example, red blood, white blood cell, muscle cell, cancer cell, fibroblasts, epithelial cell, and the like.
Number of nucleuses and/or number of cell organelles and/or distribution of cell organelles within the cell.

In an example, a cell phenotype features includes size and/or stain intensity of cancer cells expressing a certain biomarker that indicates suppressed immune cell activity. In another example another cell phenotype features includes size and/or stain intensity of cancer cells expressing checkpoint inhibitor antigen biomarker that suppresses immune cell activity.

The extracted cell phenotype features, and/or extracted cluster phenotype features, and/or extracted cluster-to-cluster features and/or other structural features and/or other spatial features may be handcrafted features, and/or features that are automatically extracted by extracting code. The features may be extracted by a machine learning (ML) model, for example, one or more or combination of neural networks, support vector machines (SVM), decision trees, boosting, random forest, and the like.

The structure (e.g., cell type) segmentations are optionally clustered according to one or more clustering requirements, to create multiple clusters. Alternatively, some structure (e.g., cell type) segmentations are clustered, and some structure (e.g., cell type) segmentations are not clustered (e.g., may be considered as a cluster only of the respective cell type segmentation). Alternatively, no clusters are created. In an implementation where no clusters are created, the clustering requirement may define that each cluster includes only the respective structure (e.g., cell type) segmentation. I.e., each structure (e.g., cell type) segmentation may be considered its own individual cluster (for further processing as described herein), which effectively defines that no clustering is performed, i.e., there are no clusters with two or more members.

Optionally, all structure (e.g., cell type) segmentations of a same cluster meet a common clustering requirement. Exemplary clustering requirement includes, but is not limited to one or more of:
According to respective cell types of the cell type segmentations, i.e., all cell type segmentations of the same cluster are of the same cell type, for example, all T cells are clustered together, all cancer cells are clustered together.
According to a combination of cell types, e.g., all T cells and B cells are clustered together, or all macrophages and blood vessel cells are clustered together, or immune cells (of a general immune cell type, or a specific immune cell type such as T cell, B cell, macrophage) and cancer cells are clustered together, and/or all immune cells are clustered together.
According to the cell phenotype features. I.e., all cell type segmentations having the same cell phenotype feature are included in a common cluster, for example, all cell type segmentations having a visible nucleus larger than a certain size (e.g., threshold) in the H&E stain are in a common cluster.
According to a combination of cell phenotype features, for example, all cell type segmentations having both a nucleus larger than a certain size and have PD-L1 positive staining are in a common cluster.
Relative location within the image. For example, all cell type segmentations located within a circle of a certain diameter are in a common cluster.
Location of the cell type segmentation relative to the region type segmentation, for example, all immune cells located within the tumor region are in one cluster, all immune cells located on the border of the tumor region are in another cluster, and all cancer cells within blood vessels are in yet another cluster.
Based on one or more items described above, for different types of structures.

Optionally, each segmentation (i.e., cell type and/or region type and/or other structure) is associated with a location coordinates, for example, x, y (and/or z) Cartesian and/or polar coordinates within the image and/or slide. The coordinates may be used to compute the relative locations, locations, and/or distances described herein.

Alternatively or additionally, the region type segmentations are clustered according to one or more clustering requirements, to create multiple clusters, for example, by type of region and/or combination of regions. For example, all region type segmentations depicting a tumor/cancer region are clustered into a common cluster. In another example, region type segmentations depicting blood vessels within a tumor region are clustered into a common cluster. Alternatively, the region type segmentations are not clustered.

Alternatively or additionally, the structure (e.g., cell type) and region type segmentations are clustered according to one or more clustering requirements, to create multiple clusters. Each cluster may include both structure (e.g., cell type) and region type segmentations.

One or more cluster phenotype features may be extracted for each of the clusters. The cluster phenotype features may be computed from an aggregation of the segmentations (e.g., cell type and/or region type and/or other structure) which are members of the respective cluster.

Exemplary cluster phenotype features include, but are not limited to one or more or combination of:
A number of segmentations (e.g., cell type and/or region type) members of the respective cluster, i.e., number of cell type segmentation members in the T cell cluster.
An average size and/or distribution of segmentation members (e.g., cell type and/or region type) of the respective cluster, optionally based on cell phenotype features of members within the respective cluster, for example, for a cluster of immune cells, the number of cells of each type of immune cell (e.g., number of T cells, number of macrophages, and the like).
An average location and/or location distribution and/or density of segmentations (e.g., cell type and/or region type) that are members of the respective cluster within the image.
An average intensity and/or intensity distribution and/or density of at least one biomarker of the segmentations (e.g., cell type and/or region type) members of the respective cluster, for example, average intensity and/or distribution of intensity of a certain biomarker within members of the respective cluster. In another example, number of segmentation members that depict each type of biomarker.
According to cell phenotype features, which are different than the cell phenotype features that were used to create the clusters (since all members of the cluster have the same such cell phenotype feature used to create the cluster).
Based on one or more items described above, for different types of structures.

Optionally, one or more cluster-to-cluster features are extracted for different combinations of two or more structure (e.g., cell) clusters. For example, cluster-to-cluster features may be computed for a pair of clusters, and/or cluster-to-cluster features may be computed for a set of three or more clusters. Multiple combinations of two or more clusters may be considered.

Exemplary cluster-to-cluster features include, but are not limited to:
Physical distance between clusters, for example, between the closest points of the clusters, between the furthest points of the clusters, between the center (e.g., center of mass) of the clusters.
Statistical distance between clusters. For example, computed using a statistical comparison function, such as a function that compares statistical distance between distributions.
Similarity between clusters. For example, computed using a similarity comparison function, such as a function that compares statistical similarity between distributions.
Differences between clusters. For example, computed using a difference comparison function, such as a function that compares statistical difference between distributions.

Cluster-to-cluster features may be computed using the respective cluster phenotype features of the two clusters, for example, statistical distance and/or similarity and/or difference between the two clusters using one or more respective cluster phenotype features of each of the clusters. In an example, statistical distance and/or similarity and/or difference between a first distribution of a certain biomarker intensity within a first cluster and a second distribution of the same certain biomarker within a second cluster. In another example, statistical distance and/or similarity and/or difference between a distribution of location of T cells within a cancer region, and distribution of location of T cells external to the cancer region.

The spatial features described herein may include, and/or be computed from, one or more of: the cluster-to-cluster features, the structure (e.g. cell) phenotype features, cluster phenotype features, and/or features of other structures optionally segmentations thereof.

Optionally, a feature vector is computed. The feature vector may be associated with each respective segmentation (i.e., cell type and/or region type and/or other structure) and/or with each respective cluster. Optionally, the feature vector is only for the structure (e.g., cell type) segmentations. Alternatively, the feature vector is only for the region type segmentations. Alternatively or additionally, the feature vector is for the computed clusters. Alternatively, there are two or more types of feature vectors, for example, one feature vector type for the structure (e.g., cell type) segmentations, and/or another feature vector type for the region type segmentations and/or another feature vector type for the clusters. Alternatively, the feature vector is a combination of two or more of the structure (e.g., cell type) segmentation, the region type segmentations, and the clusters.

The feature vector may include one or more of: the structure (e.g., cell) phenotype features extracted for the respective segmentation, an indication of a location of the respective segmentation (e.g., cell type, other structure) relative to one or more other segmentation types (e.g., region type, another cell type segmentations of another cell type, another structure), a physical distance between the respective segmentation and the one or more other segmentation types, the cell cluster phenotype features of the cluster of the respective segmentation, and/or the cluster-to-cluster features of the cluster of the respective cell type segmentation.

The cell-graph may be computed (i.e., created) based on the feature vectors of the segmentations.

Optionally, the cell-graph includes only the structure (e.g., cell type) segmentations. Alternatively, the cell-graph includes only the region type segmentations. Alternatively, the cell-graph includes only the computed clusters. Alternatively, two or more cell-graphs are created, one cell-graph type for the structure (e.g., cell type) segmentations, and/or another cell-graph type for the region type segmentations, and/or another graph for the clusters. Alternatively, the cell-graph includes a combination of the structure (e.g., cell type) segmentation, the region type segmentations, and the clusters.

Each node of the cell-graph denotes one or more of: a respective structure (e.g., cell type) segmentation, a respective region type segmentation, and a respective cluster. Each node corresponds to the assigned (e.g., associated) feature vector. Edges of the cell-graph, that connect nodes of the cell-graph, may represent a physical distance between the respective segmentation (i.e., cell type and/or region type and/or cluster) corresponding to the respective node. In region type the physical distance may be computed, for example, as a shortest distance between the regions and/or distance between region centers. In clusters the physical distance may be computed, for example, by physical distance between cluster centers. The physical distance may be: between structure (e.g., cell type) segmentations and region type segmentations corresponding to the respective nodes, between region type segmentations and region type segmentations corresponding to the respective nodes, between structure (e.g., cell type) segmentations and structure (e.g., cell type) segmentations corresponding to the respective nodes, between computed clusters representing respective nodes. The physical distance may be measured, for example, in micrometers based on the actual distance on the slide, in pixels of the image, and/or other units.

The graph may be created, for example, by linking K nearest neighbor nodes, and/or nodes up to a predefined distance.

Spatial features described herein may be extracted from the graph, for example, computed from physical distances denoted by the graph, and/or the raw physical distances may be used as the spatial features.

At 208, spatial features are extracted from the image(s) of slide(s) depicting the portion of the target tissue of the individual.

Spatial features may be, for example, hand crafted features, and/or automatically computed features such as obtained by feeding the image into a neural network and extracted data from one or more internal layers of the neural network such as feature maps and/or weights.

Spatial features may be extracted from the patches, such as per patch.

Examples of extracted spatial features include, but are not limited to: physical distance between pairs of different expressed structures, density of physical distance between pairs of different expressed structures, relative distances between pairs of different expressed structures, and relative densities between pairs of different expressed structures.

Spatial features may be extracted from the clusters, such as per cluster, and/or using the data of the clusters. Exemplary spatial features include: physical distance between clusters of different cell types, physical distance between clusters of different combinations of expressed structures, physical distance between a specific cell type and a specific expressed structure, and physical size of one or more clusters.

Optionally, the spatial features are extracted by creating the cell-graph (as described herein) based on the feature vectors of the cell type segmentations and/or clusters. Each node of the graph denotes a respective cell type segmentation and/or respective cluster. Each node includes an associated corresponding feature vector. Edges of the graph represent a physical distance between cell type segmentations and/or clusters corresponding to the respective nodes.

The graph may include nodes each denoting a respective region type segmentation. Edges of the graph may represent a physical distance between cell type segmentations and/or region type segmentations corresponding to the respective nodes, and/or between region type segmentations corresponding to the respective nodes.

The spatial features described herein may include, and/or be computed from, one or more of: the cluster-to-cluster features, the structure (e.g. cell) phenotype features, cluster phenotype features, and/or features of other structures optionally segmentations thereof.

Spatial features described herein may be extracted from the graph, for example, computed from physical distances denoted by the graph, and/or the raw physical distances may be used as the spatial features.

At 210, the spatial features are fed into the machine learning model.

Optionally, when additional data (of the individual from which the tissue depicted in the image of the slide) is available (e.g., manually entered, extracted from a record such as an electronic health record of the individual), a combination of personal data of the individual and the spatial features are fed into the machine learning model.

Optionally, when the extracted spatial features are in a cell-graph, the cell-graph is fed into a graph neural network implementation of the ML model. The graph neural network may be trained on records each including a sample graph computed from a respective sample image.

Optionally, the image(s) is fed into the ML model in combination with the spatial features.

At 212, an outcome of a target medical state may be obtained from the machine learning model.

At 214, one or more target spatial features from of spatial features that were fed into the ML model are obtained by applying an interpretability model to the machine learning model. The target spatial feature are inputted features that statistically significantly contributed to the outcome of the ML model, for example, the most significant features, and/or feature having a statistically significance to the outcome as defined by a requirement (e.g., threshold). Other interpretability models include, for example, feature maps (e.g., heat maps) extracted from internal layers of a neural network, Shapley values, and the like.

Exemplary target spatial features include, but are not limited to:
An indication of high abundance of a specific structure, for example, statistically significantly (e.g., relative to a threshold) higher than seen in other samples.
An indication of a low abundance of a specific structure, for example, statistically significantly (e.g., relative to a threshold) lower than seen in other samples.
Spatially close proximity between at least two expressed structures (e.g., closer than other pairs of expressed structures, closer than seen in other samples).
Spatially large distance between at least two expressed structures (e.g., higher than other pairs of expressed structures, higher than seen in other samples).

At 216, one or more expressed structures corresponding to the target spatial feature(s) are identified. In an exemplary implementation, two expresses structures corresponding to each target spatial feature are identified.

In the case of high/low abundance, one or more first expressed structures (e.g., protein) may be identified which are to be respectively reduce/increased, and a second expressed structure (e.g., tumor cell) where the first specific structure is to be reduced/increased.

In the case of spatially close proximity and/or spatially large distances, two (or more) expressed structures may be identified, such as the pair of structures that are spatially close and/or spatially far apart.

Examples of the expressed structure(s) include, but are not limited to: expressed protein, cell type, genomic structures, RNA, DNA, methylation, transcriptomic structure, visually distinguishable structure identified based on a staining of the slide based on immunohistochemistry and/or multiplexed immunohistochemistry and/or based on spatial transcriptomics and/or single cell analysis, and/or visually distinguishable structure identified based on immunofluorescence.

At 218, one or more features described with reference to 204-216 may be iterated.

Iterations may be performed, for example, for multiple images of multiple target tissues of multiple individuals, such as individuals suffering from a same specific medical state (e.g., medical condition, medical disease, cancer, cancer type). The images may be fed into the same ML model, which may be trained on sample images of sample slides of sample subjects with the specific medical state. The iterations may be performed for obtaining multiple candidate spatial features.

The candidate features may be analyzed to identify the target spatial features. For example, the biological impact of each candidate feature may be explored, for example, toxicity, safety, efficacy, side-effects, feasibility of manufacture, and the like. The most promising candidate spatial features (e.g., in terms of biological impact) may be selected as the spatial features used to create the drug, for example, according to a ranking and/or other scoring mechanism (e.g., assign score according to biological feasibility).

At 220, a drug to treat a target subject may be designed to target the identified expressed structure(s).

The drug may be designed for treating the specific medical state (e.g., as described with reference to 218).

Exemplary approaches for designing drugs include, but are not limited to:
The drug may be designed to engage the two (or more) expressed structures corresponding to the target spatial feature, such as to reduce the distance between the expressed structures. Such drug may be designed based on a bi-specific antibody drug family which includes two (or more) binding arms that are designed to bind targets of the two expressed structures, for example, as described with reference to Siwei Nie, Zhuozhi Wang, Maria Moscoso-Castro, Paul D'Souza, Can Lei, Jianqing Xu, Jijie Gu, Biology drives the discovery of bispecific antibodies as innovative therapeutics, Antibody Therapeutics, Volume 3, Issue 1, January 2020, Pages 18-62, https://doi(dot)org/10.1093/abt/tbaa003, and/or Labrijn, A. F., Janmaat, M. L., Reichert, J. M. et al. Bispecific antibodies: a mechanistic review of the pipeline. Nat Rev Drug Discov 18, 585-608 (2019). https://doi(dot)org/10(dot)1038/s41573-019-0028-1.
When the target medical state indicates sensitivity to immunotherapies, the drug may be designed as a bi-specific antibody and/or tri-specific antibody that targets and/or engages the identified two (or more) expressed structures corresponding to the target spatial feature. For example, the drug may be based on Bi-specific T-cell engagers (BiTEs).
The drug may be designed to disengage the two (or more) expressed structures corresponding to the target spatial feature, such as to increase the distance between the expressed structures.
When the target medical state indicates resistance to immunotherapies, the drug may be designed to block and/or disengage between the identified two (or more) expressed structures corresponding to the target spatial feature. For example, the drug may be a monoclonal antibody.
When the target spatial feature indicates low abundance of a first expressed structure (e.g., protein) within a second expressed structure (e.g., tumor cell), the drug may be designed to increase abundance of the first structure within the second expressed structure. For example, the drug may be a bi-specific monoclonal antibody having a first arm that binds the first expressed structure (e.g., protein) for increase thereof and a second arm that binds to the second expressed structure (e.g., tumor cell) where the first structure is to be increased.
When the target spatial feature indicates of high abundance of a first expressed structure (e.g., protein) located within and/or in proximity to a second expressed structure denoting a cancer and/or tumor microenvironment, where the high abundance of the first expressed structure is in comparison to normal tissue, the drug may be is designed to activate in the presence of the cancer and/or tumor microenvironment, for example, BITE, and/or other drugs designed to activate structures (e.g., T cells) to initiate tumor killing at the site of the tumor.

At 222, the created drug may be administered to treat a target subject, optionally to treat the specific medical condition (e.g., specific disease) in the target subject.

Referring now back to FIG. 3, at 302, one or more images of one or more sample slides depicting at least a portion of a sample tissue of a sample subject are provided (e.g., obtained and/or accessed), for example, as described with reference to 204 of FIG. 2.

At 304, the image(s) may be pre-processed, for example, as described with reference to 206 of FIG. 2.

At 306, one or more spatial features are extracted from the images, for example, as described with reference to 208 of FIG. 2.

At 308, additional data of the subject may be provided (e.g., obtained and/or accessed), for example, as described with reference to 202 of FIG. 2.

At 310, a medical state of the sample individual is provided (e.g., obtained and/or accessed), for example, manually entered by a user and/or automatically extracted from a record (e.g., electronic health record). Examples of medical states are described for example, with reference to 202 of FIG. 2.

At 312, a record is created. The record includes the spatial features, and optionally includes the additional data. The medical state is designated as ground truth.

At 314, features described with reference to 302-312 are iterated, to create multiple records. The multiple records are arranged into a training dataset. The records may be of a common parameter, for example, of individuals with a specific medical disease, for example, a specific cancer.

At 316, the ML model is trained on the training dataset, for example, as described with reference to 202 of FIG. 2.

Referring now back to FIG. 4, at 402, images of slides may be registered, for example, as described with reference to 206 of FIG. 2.

At 404, structures such as cells, and/or regions, are segmented, for example, as described with reference to 206 of FIG. 2.

At 406, hand crafted features, which may include spatial features and/or that are used to compute spatial features, are extracted, for example, as described with reference to 208 of FIG. 2.

Alternatively or additionally, at 408, protein expressions and/or other identified structures (e.g., based on staining) may be aggregated, for example, as described with reference to 206 of FIG. 2.

At 410, clustering is performed, for example, for similar types of structures, cells, and/or other approaches, for example, as described with reference to 206 of FIG. 2.

At 412, the extracted hand crafted features, and/or spatial features computed from the extracted hand crafted features may undergo a dimensionality reduction process, for example, as described with reference to 208 of FIG. 2.

Alternatively or additionally, at 414, the image is fed into a deep learning network for dimensionality reduction, and/or for extraction of spatial features (e.g., from hidden layers of the neural network, such as in the format of feature maps), for example, as described with reference to 208 of FIG. 2.

Alternatively or additionally, at 416, the cell-graph described herein is computed, for example, as described with reference to 206 of FIG. 2. Spatial features may be extracted from the cell-graph, as described herein, for example, as described with reference to 208 of FIG. 2.

Optionally, at 418, clinical data of the subject whose tissue is depicted in the image(s) of the slide(s) is provided, for example, clinical outcomes, medical condition, medical state, administered treatment, and the like, for example, as described with reference to 202 and/or 204 of FIG. 2.

Alternatively or additionally, at 420, additional data of the subject is provided, for example, demographic and/or omics data, for example, as described with reference to 202 and/or 204 of FIG. 2.

At 422, the extracted spatial features (e.g., from 412-416) and optionally the additional data (e.g., from 418-420) are fed into a target discovery model (e.g., as described with reference to 210), optionally a machine learning model (e.g., as described with reference to 202 of FIG. 2), for identifying one or more target spatial features, such as by applying an interpretability model to the ML model (e.g., as described with reference to 214 of FIG. 2). A drug may be designed to target two (or more) expressed structures corresponding to the identified target spatial feature(s) (e.g., as described with reference to 220 of FIG. 2).

Referring now back to FIG. 5, at 502, images of slides of subject(s) are obtained, for example, as described with reference to 204 of FIG. 2.

At 504, clinical data of the subject(s) may be obtained, for example, as described with reference to 204 of FIG. 2.

At 506, omics data of the subject(s) may be obtained, for example, as described with reference to 204 of FIG. 2.

At 508, spatial features extracted from the images (e.g., as described with reference to 208 of FIG. 2), and optionally the clinical and/or omics data are fed into a clinical outcome prediction model (e.g., as described with reference to 210 of FIG. 2), optionally a machine learning model (e.g., as described with reference to 202 of FIG. 2). A clinical outcome is obtained from the ML model (e.g., as described with reference to 212 of FIG. 2)

At 510, immune patterns, i.e., target spatial feature(s), associated with the clinical outcome are detected, such as by applying an interpretability model to the ML model (e.g., as described with reference to 214 of FIG. 2). A drug may be designed to target two (or more) expressed structures corresponding to the identified target spatial feature(s) (e.g., as described with reference to 220 of FIG. 2).

Referring now back to FIG. 6, at 602 whole slide images of a tissue biopsy of one or more subjects are imaged, for example, as described with reference to 204 of FIG. 2.

At 604, clinical and/or omics data of the subject(s) are provided, for example, as described with reference to 204 of FIG. 2.

At 606, one or more spatial features associated with response to immunotherapy are identified, for example, as described with reference to 208-218 of FIG. 2.

At 608, the identified spatial feature indicates a low proximity between proteins. At 610, one or more drugs are designed to engage between the proteins, such as BiTE drugs, for example, as described with reference to 220 of FIG. 2.

At 612, the identified spatial feature indicates a high proximity between proteins. At 614, one or more drugs are designed to dis-engage between the proteins, such as monoclonal antibodies, for example, as described with reference to 220 of FIG. 2.

At 616, the identified spatial feature indicates a high (or alternatively, low) abundance of a specific protein in the tissue. At 618 one or more drugs are designed to decrease (or alternatively, to increase) abundance of protein in the tissue, such as bi-specific antibody drugs, for example, as described with reference to 220 of FIG. 2.

Various non-limiting embodiments of the invention are also provided in Appendix 1.

In some embodiments, the images comprise tissue. In some embodiments, the images comprise cells. In some embodiments, the cells are healthy cells. In some embodiments, the tissue is healthy tissue. In some embodiments, the cells are diseased cells. In some embodiments, the tissue is diseased tissue. In some embodiments, the tissue comprises diseased and healthy cells. In some embodiments, the tissue is a tumor. In some embodiments, the tissue is from an organ. In some embodiments, the organ is selected from skin, muscle, heart, liver, pancreas, lung, brain, kidney, intestine, stomach, esophagus, lymph nodes, testes, ovary, urogenital tract, colon, gallbladder, and glands. In some embodiments, the tissue is fixed. In some embodiments, the tissue is permeabilized.

In some embodiments, the images are of bodily fluid. In some embodiments, the bodily fluid is selected from at least one of: blood, serum, plasma, gastric fluid, intestinal fluid, saliva, bile, tumor fluid, breast milk, urine, interstitial fluid, cerebral spinal fluid and stool. In some embodiments, the fluid is blood.

As used herein, the term “biological components” refers to unique molecules or structure that can be identified in a biological sample. In some embodiments, the biological components are identifiable biological components. In some embodiments, the biological components are unique. In some embodiments, identifiable is uniquely identifiable. In some embodiments, the components, are molecules. In some embodiments, the components are organic. In some embodiments, components are structures. As used herein, “biological” refers to a component that can from a living organism. In some embodiments, the components are cell associated. In some embodiments, the components are extracellular. In some embodiments, the components are soluble.

In some embodiments, the component is a protein. In some embodiments, the component is a nucleic acid molecule. In some embodiments, the nucleic acid molecule is DNA. In some embodiments, the nucleic acid molecule is RNA. In some embodiments, the RNA is an mRNA. In some embodiments, the RNA is a regulatory RNA. Examples of regulatory RNAs include, but are not limited to ribosomal RNAs, microRNAs, small interfering RNAs, short hairpin RNAs, pi RNAs and the like. In some embodiments, the component is a complex. In some embodiments, the component is chromatin. In some embodiments, the component is a lipid. In some embodiments, the component is an ion. In some embodiments, the component is a macromolecule. In some embodiments, the component is an organelle. Examples or organelles include, but are not limited to membranes (e.g., plasma membrane, nuclear membrane, mitochondrial membrane), ribosomes, nuclei, nucleoli, mitochondria, ER, lysosomes, endosomes, lipid rafts, speckles and centromeres.

In some embodiments, the images comprise at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 70, 75, 80, 85, 90, 95, 100, 150, 200, 250, 300, 400 or 500 biological components. Each possibility represents a separate embodiment of the invention. In some embodiments, the images comprise at least 20 biological components. In some embodiments, the images comprise at least 50 biological components. In some embodiments, the images comprise at least 60 biological components. It will be understood by a skilled artisan that the number of components will be limited only by the number of channels that can be imaged in one image.

In some embodiments, the components are identifiable. In some embodiments, the components are stained. The staining of proteins and organelles are well known in the art. Immunohistochemistry, immunofluorescence and the like may be used for protein staining. Stains specific to organelles, lipids, pHs and other conditions and structures are also well known and may be employed. Nucleic acid molecules can also be identified by staining as DNA dyes and anti-DNA antibodies are well known. Additionally, tagged primers/probes may be employed. Further, methods of spatial transcriptomics are well known in the art and allow for the determination of the unique spatial position of nucleic acid molecules without tags but rather by sequencing of the molecules. In some embodiments, the molecules comprise a barcode or unique molecular identifier (UMI) that allows for determining of spatial position. In some embodiments, the sequencing identifies the barcode or UMI. In some embodiments, the sequencing determines the spatial position.

In some embodiments, the images are two-dimensional. In some embodiments, the images are three-dimensional. Methods of two dimensional and three-dimensional imaging are well known in the art and any such method may be used. In some embodiments, the image is a three-dimensional rendering of a cell. In some embodiments, the image is a three-dimensional rendering of a tissue. In some embodiments, the image is a three-dimensional rendering of an organ or portion thereof. In some embodiments, the image is a three-dimensional rendering of an organoid or portion thereof.

It will be understood that when two or more components are closer to each other or more in contact or more abundant in a preferred state than in a not preferred state than the agent will bring the two or more components into association (cause association) or greater association, or greater contact or increase abundance in the region of the other component. In some embodiments, association is binding. This can be achieved in numerous ways envisioned by the invention. For example, a multi-specific agent can bind the at least two components. This agent would thus bring the at least two components into close proximity and therefore into association. In some embodiments, association is proximity. In some embodiments, association is in contact. In some embodiments, such an agent is an engager. In some embodiments, the agent brings the at least two components into engagement. In some embodiments, the agent increases the density or abundance of one of the components in the vicinity of the other. This can be done via a multi-specific agent as well or by a mono-specific agent (e.g., an antibody). By binding the lowly dense or abundant component the agent can increase its presence. Alternatively, the multi-specific agent will bind one component from a distant location, for example another tissue or another part of the body and bring it to the desired location by binding to the other component.

It will be further understood that when two or more components are farther apart from each other or less in contact or less abundant in a preferred state than in a not preferred state than the agent will disrupt association, inhibit binding, pull away one of the components or otherwise block the association. This can be achieved by means well known in the art, such as blocking antibodies, antagonists or multi-specific antibodies.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

It is expected that during the life of a patent maturing from this application many relevant machine learning models will be developed and the scope of the term machine learning model is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

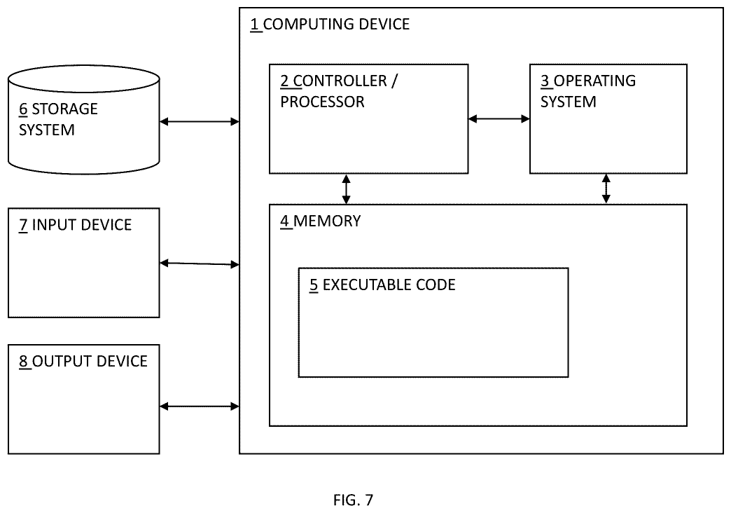

Reference is now made to FIG. 7, which is a block diagram depicting a computing device, which may be included within an embodiment of a system for designing a drug, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may designing a drug as further described herein. Although, for the sake of clarity, a single item of executable code 5 is shown in FIG. 7, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to slide images taken from subjects or patients may be stored in storage system 6 and may be loaded from storage system 6 into memory 4 where it may be processed by processor or controller 2. In some embodiments, some of the components shown in FIG. 7 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 7) such as one or more CPUs or graphics processing units (GPUs), or a dedicated hardware device may perform the relevant calculations.

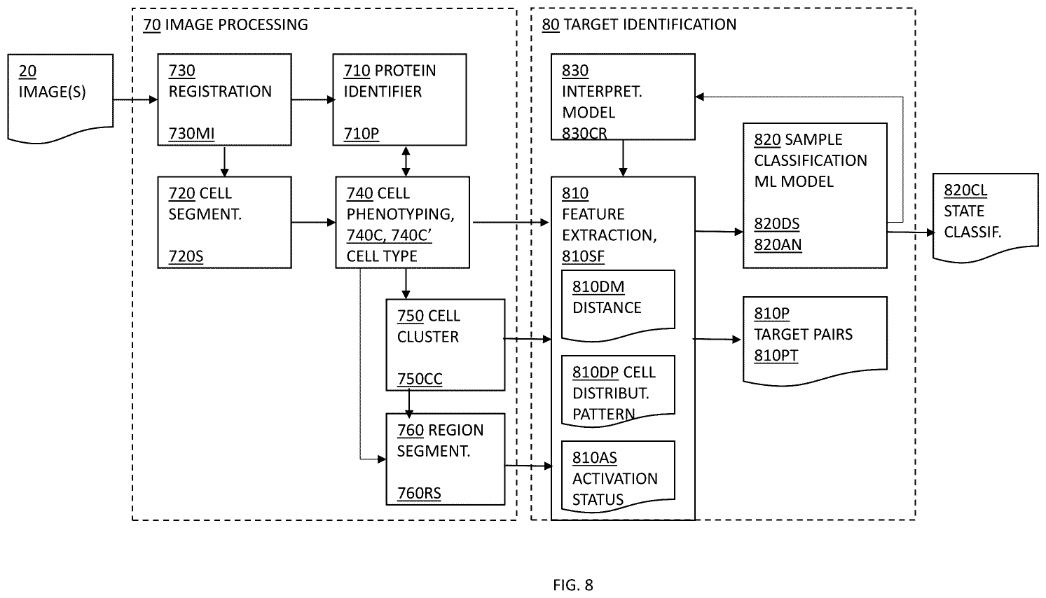

Reference is now made to FIG. 8, which depicts a system 100 for designing a drug, according to some embodiments of the invention.

According to some embodiments of the invention, system 100 may be implemented as a software module, a hardware module, or any combination thereof. For example, system may be or may include a computing device such as element 1 of FIG. 7, and may be adapted to execute one or more modules of executable code (e.g., element 5 of FIG. 7) to design a drug, as further described herein.

System 100 may be the same as system 200 depicted in FIG. 1.

Additionally, or alternatively, system 100 may be configured to implement the steps and methods depicted in the flow diagrams of FIG. 2 and/or FIG. 3.

Additionally, or alternatively, system 100 may be implemented by, or include the modules of FIG. 4.

As shown in FIG. 8, system 100 may be schematically divided into two main subsystems: An image processing subsystem 70 may be adapted to receive one or more images 20, and analyze them as explained herein, to facilitate extraction of specific features therefrom. A target identification subsystem 80 may further analyze the obtained features 810, to gain insight on a subject's condition, and/or suggest specific target proteins for use in designing a drug for treating such subjects.

Arrows in FIG. 8 may represent flow of one or more data elements to, and from system 100 and/or among modules or elements of system 100. Some arrows have been omitted in FIG. 8 for the purpose of clarity.

As shown in FIG. 8, image processing subsystem 70 of system 100 may obtain at least one image 20 representing protein expression in at least one sample, taken from at least one respective subject. For example, image 20 may include one or more slide images sliced and dyed to exhibit location of proteins in a sample (e.g., a biopsy) taken from a patient.

Additionally, or alternatively, image 20 may be, or may include a multiplexed image, where different protein types are uniquely stained.

In another example, system 100 may receive a plurality of slide images 20, e.g., representing a tissue section, where protein types in each slide are uniquely stained. Image processing subsystem 70 may include a registration module 730, which may be the same as registration module 402 of FIG. 4. Registration module 730 may be configured to register the plurality of slide images 20 with each other, to produce a multiplexed image 730MI of the tissue section. For the purpose of brevity “image 20” and “image 730MI” may be used herein interchangeably according to context.

As shown in FIG. 8, image processing subsystem 70 may include a cell segmentation module 720, which may be the same as segmentation 404 of FIG. 4. Cell segmentation module 720 may be configured to apply an ML based segmentation algorithm on the at least one image 20 (730MI), to obtain a plurality of cell segments 720S. Each cell segment 720S may represent a cell in the sample depicted in image 20.

Image processing subsystem 70 may also include a protein identification module 710, adapted to identify a location of specific proteins 710P in image 20 (730MI).

For example, protein identification module 710 may associate a color of a specific region in image 20 with a specific dye, used for enhancing the presentation of a respective protein, thereby identifying both a location, and a type of a protein depicted in image 20/730MI. Both the location and type of a protein may therefore be enumerated herein as 710P.

Additionally, or alternatively, image processing subsystem 70 may include a cell phenotyping module 740, adapted to collaborate with cell segmentation module 720 and protein identification module 710 so as to identify types 740C of cells that corresponds to specific cell segments 720S in image 20 (730M1).

According to some embodiments, cell phenotyping module 740 may include a deep, ML-based classifier, that may be trained (e.g., by a supervised training scheme) to predict marker positivity for each cell, and subsequently utilize the predicted marker positivity to classify cell segments 720S according to their types 740C.

Phenotyping module 740 may identify one or more cell segments as pertaining to a first cell type, or a second cell type, based on indications of location 710P of protein expression within the one or more cell segments 720S, as depicted in image 20 (730MI).

For example, protein identification module 710 may collaborate with phenotyping module 740 to identify, in image 20, a location of proteins 710P of a first type, within a first type of cells (e.g., immune cells), and identify location of proteins 710P of a second type, within a second type of cells (e.g., cancer cells).

In other words, one of the first cell type and second cell type may be a cancer cell, and the other cell type may be an immune cell. Additionally, or alternatively, one of the first cell type and second cell type may be an immune cell, and the other cell type may be a disease cell, or a healthy cell of the same cell type as said disease cell.

As elaborated herein, by analyzing proximity (or lack thereof) between location of proteins 710P of different types, in specific cell types of interest, system 100 may be utilized to gain insight regarding a condition of the sample (and relevant patient). System 100 may then be further utilized to determine specific definitions or requirements for designing efficient drugs for treating the patient's condition. 70

According to some embodiments, image processing subsystem 70 may include a clustering module 750, adapted to identify clusters of cells 750CC based on co-expression of cells. For example, clustering module 750 may be adapted to identify clusters 750CC of cells pertaining to specific types 740C.

Additionally, or alternatively, image processing subsystem 70 may include a region segmentation module 760, adapted to identify a spatial neighborhood, or a regional segment 760RS of interest, based on the identified clusters 750CC. For example, a regional segment 760RS may define a region of a specific tissue, a region of a tumor, a region of a stroma, and the like.

According to some embodiments, target identification subsystem 80 of system 100 may include a feature extraction module 810, adapted to extract, from the at least one image, a spatial feature 810SF value.

As explained herein, spatial feature 810SF may combine the cell type 740C (including marker positivity), cluster 750CC, and region/segment 760RS to calculate spatial features 810SF that capture cell distribution in areas, combined with phenotypic states, and cell-cell interactions based on distance.

Additionally, or alternatively, spatial features 810SF may represent a spatial relationship between proteins of the first type 710P (e.g., proteins of immune cell types 740C) and proteins of the second type 710P (e.g., proteins of cancer cell types 740C).

Spatial feature 810SF may represent metrics of (a) abundance, (b) density and/or (c) distribution, pertaining to (i) individual cells, (ii) groups of cells, (iii) individual proteins and/or (iv) groups of proteins depicted in images 20/730MI.

For example, spatial relationship, or spatial feature 810SF may include, for example a distance metric value 810DM, indicating a metric of distance between proteins of the first type, and proteins of the second type in image 20. In other words, spatial feature value 810SF (distance metric value 810DM) may include a measure of a distance between a protein of the first type and a protein of the second type, a distribution of distances between proteins of the first type and proteins of the second type, a contact between a protein of the first type and a protein of the second type, a structure of a protein of the first type and a protein of the second type, and the like.

In another example, spatial feature value 810SF (distance metric value 810DM) may represent properties of pairs 810P of proteins of different types. In other words, feature extraction module 810 may identify, within image 20 (730MI) a plurality of protein pairs 810P, each including a protein of the first type (e.g., a protein of an immune cell type) and a protein of the second type (e.g., a protein of an immune cell type), whose locations are within a predetermined radius or distance. Spatial feature value 810 may then include features such as an abundance of the protein pairs 810P within image 20, a distribution of pairs 810P within image 20, and the like.

In another example, spatial feature 810SF value may represent properties of a single type of proteins, including for example a distribution of proteins of the first type in image 20, a distribution of proteins of the second type in image 20, an abundance of proteins of the first type in image 20, an abundance of proteins of the second type in image 20, and the like.

In another example, spatial feature value 810SF may include a metric of cell distribution 810DP. For example, cell distribution 810DP may indicate a distance between a cell of the first type and a cell of the second type, a distribution of distances between cells of the first type and cells of the second type, a contact between a cell of the first type and a cell of the second type, and the like.

In another example, spatial feature value 810SF may include a metric of distribution of distances among different entities, such as distribution of distances between pairs of closest (e.g., neighboring) cells (of the same cell type, or of different cell types), distribution of distances between closest (e.g., neighboring) instances of proteins (e.g., of the same protein type, or of different protein types), and the like.

Additionally, or alternatively, spatial feature value 810SF may relate to a 3D structure, representing the biological sample of interest. For example, as elaborated herein, registration module 730 may receiving a plurality of slide images 20 representing a respective plurality of tissue sections, and stack the plurality of slide images to generate image 730MI as a 3D image or structure. Spatial feature 810SF may therefore represent metrics of (a) abundance, (b) density and/or (c) distribution, pertaining to (i) individual cells, (ii) groups of cells, (iii) individual proteins and/or (iv) groups of proteins, between two or more sections or layers of 3D image or structure 730MI. Examples of spatial features 810SF are brought herein (e.g., in relation to 2D images), and will not be repeated here for the purpose of brevity.

As shown in FIG. 8, target identification subsystem 80 of system 100 may include a Machine Learning (ML) based sample classification model 820. ML-based classification model 820 may be the same ML model as described with reference to 202 of FIG. 2. Classification model 820 may be the same as ML model 122A of FIG. 1. Classification model 820 may be pretrained to classify (e.g., predict a classification 820CL of) the at least one sample or subject as one of a predetermined set of subject states, based on at least one image 20/730MI and/or from data (e.g., spatial features 810SF) derived from at least one image 20/730MI.

For example, classification model 820 may (e.g., during a training stage) receive an annotated training dataset 820DS. Training dataset 820DS may be the same as training dataset 122B of FIG. 1. Training dataset 820DS may include (i) one or more images 20/730MI of tissue samples taken from subjects, and (ii) corresponding annotations 820AN or labels, indicating a state of these subjects. System 100 may then utilize any appropriate training scheme (e.g., a back-propagation scheme) to train sample classification model 820 so as to determine a state of a subject or sample based on the one or more images of the training dataset 820DS, while using annotations 820AN as supervisory information.

Additionally, or alternatively, classification model 820 may (e.g., during a training stage) receive an annotated training dataset 820DS. Training dataset 820DS may be the same as training dataset 122B of FIG. 1. Training dataset 820DS may include (i) one or more spatial features 810SF, extracted from images 20/730MI of tissue samples taken from subjects, and (ii) corresponding annotations 820AN indicating a state of these subjects. System 100 may then train sample classification model 820 to determine a state of the sample or subject, based on the one or more spatial features 810SF, while using said annotations as supervisory information.

During a subsequent (e.g., inference) stage, as described with reference to 212 of FIG. 2, system 100 may apply sample classification model 820 on one or more spatial features 810SF of target image(s) 20/730MI, to identify, or classify 820CL a state of the sample or subject as one of the predetermined set of states.

Target identification subsystem 80 may include an interpretability model 830. Interpretability model 830 may be the same as interpretability model 122D of FIG. 1.

As known in the art, an interpretability model, in the context of machine learning and artificial intelligence, may refer to a method or technique for providing insights into how a machine learning model makes predictions or decisions. This could involve identifying important features, highlighting influential factors, or providing context for a particular prediction. Interpretability model may focus on identifying which features or input variables are most influential in driving the model's predictions, using techniques such as feature importance scores, permutation importance calculation, SHAP (SHapley Additive exPlanations) values, and the like.

Interpretability model 830 may be configured to compute a correlation 830CR value, representing correlation between at least one spatial feature value 810SF and the classification 820CL of subject states.

For example, system 100 may apply interpretability model 830 (122D) on sample classification model 820 (122A), as discussed in relation to 214 of FIG. 2. Interpretability model 830 may thereby calculate correlation value 830CR as a contribution of the spatial feature value 810SF, or the spatial relationship represented by spatial feature value 810SF, to the classification of the subject's state.

Additionally, or alternatively, interpretability model 830 (122D) may employ a statistical test such as the Wilcoxon rank sum test and/or Welch's T-test as known in the art, to select spatial features 810SF.

According to some embodiments, the at least one image 20 may include a plurality of protein types, corresponding to a respective plurality of cell types. As explained herein, ML based classification model 820 may be configured to classify the sample of image(s) 20 to one of the predetermined set of subject states, based on spatial feature values 810SF of pairs 810P of protein types, derived from the at least one image 20.

In such embodiments, interpretability model 830 may be configured to identify a pair of protein types, whose spatial feature value 810SF is highly correlated (high correlation score 830CR) with classification 820, and therefore significantly contributing to classification 820 of the state of a subject.

In other words, interpretability model 830 may collaborate with feature extraction module 810 to identify statistically significant pairs of protein types 810PT. Interpretability model 830 may thereby provide one protein type of the pair 810PT as a first protein type for designing a drug, and provide another protein type of pair 810PT as a second protein type for designing the drug. Interpretability model 830 may therefore select, based on correlation 830CR, at least one of the first type of proteins and second type of proteins, to design the drug.

For example, a feature 810SF may represent a distance metric between specific protein types 810PT of a first pair of protein types 810PT (e.g., a specific immune cell protein and a specific tumor cell protein), and may be highly correlated (high correlation score 830CR) with classification 820 (e.g., resilience of a tumor). In other words, a close distance metric value feature (e.g., small 810SF value) may correspond to classification 820 of low resilience of tumor cells. In such embodiments, interpretability model 830 may select at least one of (i) the tumor cell protein type and (ii) the immune cell protein type for designing the drug.

In another example, a feature 810SF may represent a distribution of cells of a specific type, having specific protein types 810PT. In other words, feature 810SF may represent a distance metric between cells of the same type (e.g., a specific immune cell type or a specific tumor cell type), having specific protein types 810PT.

Additionally, or alternatively, feature 810SF may represent a fraction of cells of the same type, having specific protein types 810PT, in the same spatial area compartment or in close proximity.

Feature 810SF (cell distribution or cell distance metric) may be highly correlated (e.g., have high correlation score 830CR) or differentially expressed with classification 820 (e.g., resilience of a tumor). For example, feature 810SF that represents a close distance between cells of the tumor (e.g., small 810SF value) may represent cell distribution that characterizes tumor-cell clusters. Feature 810SF may therefore correlate with classification 820 of high resilience (low responsiveness) to treatment. In such embodiments, interpretability model 830 may refrain from selecting (or omit a selection of) at least one of (i) the tumor cell protein type and (ii) the immune cell protein type as target for designing the drug.

Additionally, or alternatively, interpretability model 830 may be configured to provide a list of pairs of protein types 810PT, ordered by the magnitude of the contribution of the spatial feature value 810SF of each pair of protein types 810PT to the classification 820CL of the state of a subject, for usage in designing the drug.

According to some embodiments, the drug may be bispecific, in a sense that it may simultaneously relate to the two proteins of protein pair type 810PT. Additionally, or alternatively, the drug may be adapted to modulate an interaction between proteins of the first type and proteins of the second type in pair 810PT. In this context, the term “modulate” may indicate that the designed drug may be configured to change (e.g., increase or decrease) a relation, affinity, or interaction between the proteins of the first type and proteins of the second type in pair 810PT, according to a desired application.

For example, spatial feature 810SF (e.g., distance metric 810DM) may indicate a distance (e.g., an average distance) between proteins of the first type and proteins of the second type in pair 810PT that surpasses a predetermined threshold. The designed drug may be configured to modulate the interaction between proteins of the first type and proteins of the second type by associating or connecting between proteins of the first type and proteins of the second type.

In another example, spatial feature 810SF (e.g., distance metric 810DM) may indicate a distance (e.g., an average distance) that is below a predetermined threshold. The designed drug may be configured to modulate the interaction between proteins of the first type and proteins of the second type by disassociating, disrupting, or blocking a connection between proteins of the first type and proteins of the second type in pair 810PT.

Additionally, or alternatively, system 100 may incorporate additional information and restrictions in the suggestion of pairs of cell types 810PT for designing the drug.

For example, cell phenotyping module 740 may determine a condition, or sub-type 810AS of specific cell types 740C, to avoid suggesting or selecting specific cell type pairs 810PT for designing the drugs.

For example, the first cell type 740C may be an immune cell type and the second cell type may be a cancerous cell. Embodiments of the invention may typically suggest selection of a pair of proteins, one from each cell type, to be used in designing a bispecific drug that may bring cells of the two cell types together. However, system 100 may also negate selection of such a pair of proteins, given identification of specific conditions or subtypes 810AS of the immune cells.

In other words, system 100 may identify one or more cell segments as respective immune cells, belonging to the first cell type (e.g., immune cells). Based on indication of protein expression 710P within respective cell segments 720S, system 100 may determine a feature of the cell's condition 810AS such as an activation status 810AS value of cells of the first cell type (immune cells). For example, system 100 may determine that the immune cells in image 20 are too old to be efficiently targeted against the corresponding cancer cells. System 100 may then select (or avoid from selecting) at least one of the first type of proteins (e.g., of the immune cells) and second type of proteins (e.g., of the cancer cells) further based on the determined cell activation status value 810AS.

Additionally, or alternatively, system 100 may suggest cell type pairs 810PT further based on spatial features of cell distribution 810DP.

For example, the first cell type 740C may be an immune cell type and the second cell type may be a cancerous cell. As explained herein, phenotyping module 740 may identify a plurality of cell segments 720S as respective cancer cells, belonging to the second cell type. Feature extraction module 810 may subsequently define cell distribution 810DP as a region of a cluster of cancer cells, based on this identification. The cluster of cancer cells may be characterized as having a small number (or none of) immune cells therein. System 100 may determine that such a cluster may not be efficiently treated by the immune cells, and subsequently select (or avoid from selecting) at least one of the first type of proteins (of the immune cells) and second type of proteins (of the immune cells) further based on the defined cluster of cancer cells.

Additionally, or alternatively, feature extraction module 810 may define cell distribution 810DP as a region of a cluster of immune cells. The cluster of immune cells may be characterized as having a small number of cells other types therein. System 100 may determine that such immune cells may not efficiently be used to treat tumors, and subsequently select (or avoid from selecting) at least one of the first type of proteins (of the immune cells) and second type of proteins (of the immune cells) further based on the defined cluster of immune cells.

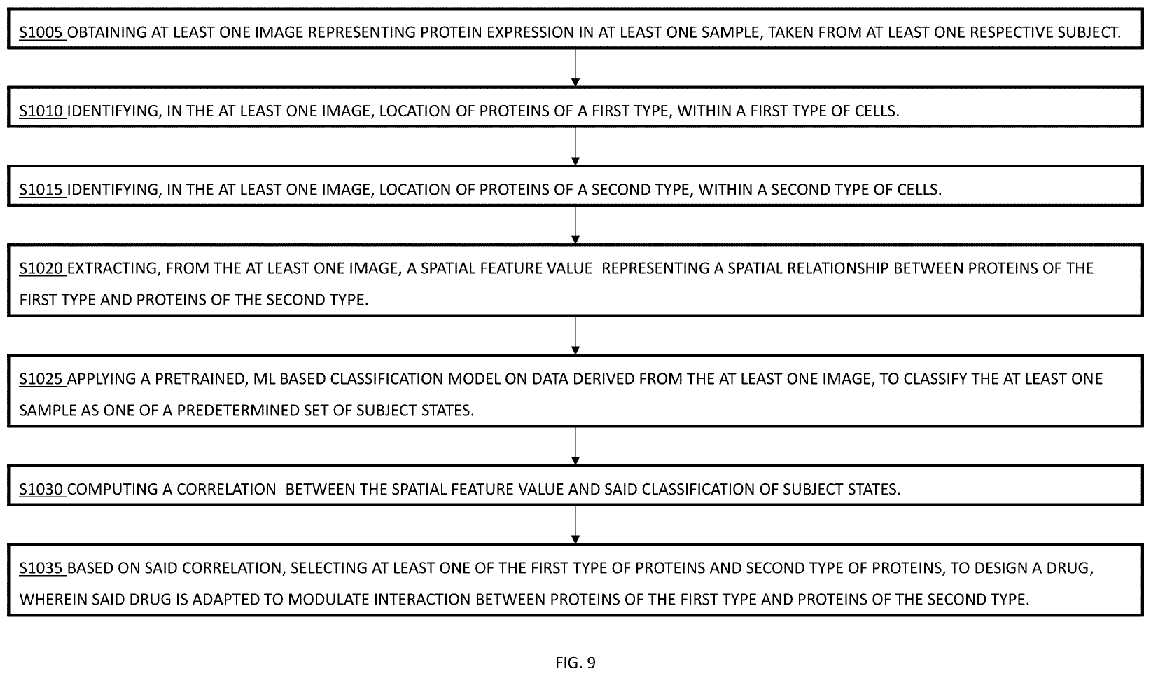

Reference is now made to FIG. 9, which is a flow diagram depicting a method of designing a drug by at least one processor (e.g., processor 2 of FIG. 7), according to some embodiments of the invention.

As shown in step S1005, the at least one processor 2 may obtain at least one image (e.g., 20/730MI of FIG. 8) representing protein expression in at least one sample, taken from at least one respective subject.

As shown in steps S1010 and S1015, the at least one processor 2 may identify, in the at least one image 20/730MI, location of proteins (e.g., 710 of FIG. 8) of a first type, within a first type of cells (e.g., 740C of FIG. 8), and location of proteins 710 of a second type, within a second type of cells 740C.

As shown in step S1020, the at least one processor 2 may extract, from the at least one image, a spatial feature value (e.g., 810SF of FIG. 8) representing a spatial relationship between proteins of the first type 710P and proteins of the second type 710P.

As shown in step S1025, the at least one processor 2 may apply a pretrained, ML-based classification model (e.g., 820 of FIG. 8) on data derived from the at least one image 20/730MI, to classify (e.g., 820CL of FIG. 8) the at least one sample as one of a predetermined set of subject states. In some embodiments, the data derived from the at least one image 20/730MI may include a version of the image 20/730MI itself. Additionally, or alternatively, the data derived from the at least one image 20/730MI may include, for example, the one or more spatial feature values 810SF.

As shown in step S1030, the at least one processor 2 may employ an interpretability model (e.g., 830 of FIG. 8) to compute a correlation (e.g., 830CR of FIG. 8) between the spatial feature value 810SF and the classification of subject states 820CL.

As shown in step S1035, based on said correlation, the at least one processor 2 may select at least one of the first type of proteins 710P and second type of proteins 710P, to design a drug. The drug may be designed so as to modulate interaction between proteins of the first type 710P and proteins of the second type 710P.

Embodiments of the invention may provide a practical application in the field of pharmaceutical technology, by providing a novel, and inventive platform for designing drugs, based on slide images, sampled from subjects or patient having (or not having) a condition or disease of interest.

1. A method of designing a drug by at least one processor, the method comprising:
obtaining at least one image representing protein expression in at least one sample, taken from at least one respective subject;
identifying, in the at least one image, location of proteins of a first type, within a first type of cells;
identifying, in the at least one image, location of proteins of a second type, within a second type of cells;
extracting, from the at least one image, a spatial feature value representing a spatial relationship between proteins of the first type and proteins of the second type;
applying a pretrained, Machine Learning (ML) based classification model on the at least one image and/or said spatial feature value, to classify the at least one sample as one of a predetermined set of subject states;
computing a correlation between the spatial feature value and said classification of subject states; and
based on said correlation, selecting at least one of the first type of proteins and second type of proteins, to design a drug, wherein said drug is adapted to modulate interaction between proteins of the first type and proteins of the second type.

2. The method of claim 1, wherein computing a correlation between the spatial feature value and the state of the subject comprises applying an interpretability model on the sample classification model, to calculate said correlation as a contribution of the spatial relationship to the classification of the subject's state.

3. The method of claim 1, further comprising:
applying an ML based segmentation algorithm on the at least one image, to obtain a plurality of cell segments, each representing a cell in the sample; and
identifying one or more cell segments as pertaining to the first cell type or the second cell type, based on indication of protein expression within the one or more cell segments, as depicted in the at least one image.

4. The method of claim 3, wherein one of the first cell type and second cell type is an immune cell and, and wherein the other cell type is a disease cell, or a healthy cell of the same cell type as said disease cell.

5. The method of claim 3, wherein the first cell type is an immune cell type, and wherein the method further comprises:
identifying one or more cell segments as respective immune cells, belonging to the first cell type;
determining a cell activation status value of the immune cells based on indication of protein expression within the respective cell segments; and
selecting at least one of the first type of proteins and second type of proteins further based on the determined cell activation status value.

6. The method of claim 3, wherein the second cell type is a cancer cell type, and wherein the method further comprises:
identifying a plurality of cell segments as respective cancer cells, belonging to the first cell type;
defining a cluster of cancer cells based on said identification; and
selecting at least one of the first type of proteins and second type of proteins further based on the defined cluster of cancer cells.

7. The method of claim 1, wherein said spatial feature value is a measure of at least one of: a distance between a protein of the first type and a protein of the second type, a distribution of distances between proteins of the first type and proteins of the second type, a distance between a cell of the first type and a cell of the second type, a distribution of distances between cells of the first type and cells of the second type, a contact between a protein of the first type and a protein of the second type, a contact between a cell of the first type and a cell of the second type, a structure of a protein of the first type and a protein of the second type, a distribution of proteins of the first type, a distribution of proteins of the second type, an abundance of proteins of the first type, an abundance of proteins of the second type.

8. The method of claim 1, wherein the spatial relationship comprises a distance metric, indicating a distance between proteins of the first type, and proteins of the second type in the at least one image.

9. The method of claim 1, further comprising identifying, within the at least one image, a plurality of protein pairs, each comprising a protein of the first type and a protein of the second type, whose locations are within a predetermined distance, and wherein the spatial feature value is defined as an abundance of the protein pairs within the at least one image.

10. The method of claim 2, wherein the at least one image comprises a plurality of protein types, corresponding to a respective plurality of cell types, and wherein the ML based classification model is configured to classify the at least one sample to one of the predetermined set of subject states based on spatial feature values of pairs of protein types, derived from the at least one image.

11. The method of claim 10, wherein the interpretability model is further configured to
identify a pair of protein types, whose spatial feature value statistically significantly contributed to the classification of the state of a subject; and
provide one protein type of the pair as the first protein type, and the other protein type of the pair as the second protein type.

12. The method of claim 1, further comprising training the ML based classification model by:
receiving a training dataset, comprising (i) one or more images of tissue samples taken from subjects and/or (ii) one or more spatial features, extracted from images of tissue samples taken from subjects;
receiving corresponding annotations indicating a state of said subjects; and
training the classification model to determine a state of a subject based on the training dataset, while using said annotations as supervisory information.

13. The method of claim 1, wherein the at least one image is an image of a slide, containing a tissue section, and wherein obtaining the at least one image comprises:
receiving a plurality of slide images representing a tissue section, wherein protein types in each slide are uniquely stained; and
registering the plurality of slide images with each other, to produce a multiplexed image of the tissue section.

14. The method of claim 1, wherein obtaining the at least one image comprises:
receiving a plurality of slide images representing a tissue section, wherein protein types in each slide are uniquely stained; and
registering the plurality of slide images with each other, to produce a multiplexed image of the tissue section.

15. The method of claim 1, wherein the subject states are selected from a list consisting of: a healthy state, a disease state, a state of responding to therapy, a state of non-response to therapy, a state of disease regression, a state of disease stability, a state of disease progression, a state of positive disease prognosis, a state of negative disease prognosis, a state of disease resistance, and a state of disease susceptibility.

16. The method of claim 8, wherein the distance metric indicates a distance that surpasses a predetermined threshold, and wherein modulating the interaction between proteins of the first type and proteins of the second type comprises associating between proteins of the first type and proteins of the second type.

17. The method of claim 8, wherein the distance metric indicates a distance that is below a predetermined threshold, and wherein modulating the interaction between proteins of the first type and proteins of the second type comprises disrupting an association between proteins of the first type and proteins of the second type.

18. A method of drug design, the method comprising:
receiving, by a trained machine learning (ML) model, one or more images of a tissue sample of a subject, wherein said ML model is trained to distinguish between tissue samples from subjects in a first state and tissue samples from subjects in a second state;
classifying said received one or more images as being from a subject in the first state or second state by applying said trained ML model; and
extracting from said ML model groups of biological components comprising at least two biological components whose spatial relationship contributed to said classifying,
thereby identifying at least two biological components for drug design,
wherein said biological components are selected from proteins, nucleic acid molecules, lipids, ions, macromolecules and organelles.

19. The method of claim 18, wherein said spatial relationship is a measure of at least one of: distance between said at least two biological components, distribution of distances between said at least two biological components, contact of said at least two biological components, association of said at least two biological components, structure of said at least two biological components, density of said at least two biological components, abundance of said at least two biological components.

20. The method of claim 18, wherein said second state is an undesired state and said first state is a desired state, and (a) said pair of biological components are closer to each other in said second state than said first state and said drug disrupts the association of said pair of biological components; or (b) said pair of biological components are closer to each other in said first state than said second state and said drug causes association of said pair of biological components to each other.
